(19) United States
(12) Patent Application Publication
(10) Pub. No.: US 2005/0197307 A1
(43) Pub. Date: Sep. 8, 2005

(54) Compounds and methods for treating and screening viral reactivation

(75) Inventors: Shelly L. Berger, Wayne, PA (US); Nigel W. Fraser, Philadelphia, PA (US); Jeffrey J. Leary, Wayne, PA (US); Ruth Tal-Singer, Media, PA (US)

Correspondence Address: GLAXOSMITHKLINE;Corporate Intellectual Property - UW2220, P.O. Box 1539, King of Prussia, PA, 19406-0939, US

(73) Assignees: SmithKline Beecham Corporation; The Wistar Institute

(21) Appl. No.: 11/016,129

(22) Filed: Dec. 17, 2004

Related U.S. Application Data
(63) Continuation of application No. 10/108,164, filed on Mar. 26, 2002, now abandoned.
Application No. 10/108,164 is a continuation of application No. 09/424,348, filed on Nov. 22, 1999, now abandoned.
(60) Provisional application No. 60/051,633, filed on Jul. 3, 1997.
(60) Provisional application No. 60/054,515, filed on Aug. 1, 1997.
(60) Provisional application No. 60/080,352, filed on Apr. 1, 1998.

(51) Int. Cl. A61K 48/00; C12Q 1/70; C12Q 1/68; C07H 21/02; A61K 38/21

(52) U.S. Cl. 514/44; 435/5; 435/6; 536/23.1; 424/85.6; 424/85.7

(57) ABSTRACT

This invention relates to host cellular factors as therapeutic and diagnostic compounds, and methods using such factors for screening for antiviral compounds, particularly compounds useful to treat Herpes virus infections, such as HSV-1 and HSV-2 infections.

### FIELD OF THE INVENTION

This invention relates to newly identified attributes of cellular and viral polynucleotides and polypeptides, the uses of such polynucleotides and polypeptides, as well as the production of such polynucleotides and polypeptides. This invention also relates to inhibiting the biosynthesis, action or interaction of such polynucleotides and/or polypeptides and to the use of such inhibitors in therapy, particularly therapy for viral reactivation, especially Herpesvirus reactivation, as well as for prophylaxis.

### BACKGROUND OF THE INVENTION

Following primary infection, latent Herpes simplex virus (herein “HSV”) persists in sensory ganglia of the peripheral nervous system. The virus can undergo sporadic reactivation to produce recurrent mucocutaneous lesions at peripheral sites innervated by the infected ganglia (reviewed in Fraser et al., 1991, Roizman, 1991, Stevens, 1989). Reactivation stimuli range from direct mechanical or pharmacological insults to the neuron and surrounding tissue to systemic changes in immune modulators and neurotransmitters (Fraser et al., 1991, Fraser & Valyi-Nagy, 1993, Hill, 1985). The earliest molecular events in neuronal cells that trigger reactivation of HSV remain unclear. It has been suggested that these events include altered expression of cellular factors such as induction of transcriptional activators and down-regulation of repressors (Sheng & Greenberg, 1990). Identification of cellular factors which are induced during the reactivation process may lead to better understanding of the cellular environment during viral induction and should facilitate development of an effective treatment or prophylaxis of reactivation an/or infection.

The present knowledge of the molecular pathogenesis of HSV latency and reactivation was generated from studies in laboratory animals including mice, guinea pigs and rabbits (reviewed in Roizman & Sears, 1987). The Applicants and others have found current murine in vivo models to be inefficient in reactivation of the viral genome (Fawl et al., 1996, Fawl & Roizman, 1993, Harwick et al., 1987, Openshaw et al., 1979, Sawtell & Thompson, 1992, Willey et al., 1984). In contrast, the murine explant reactivation model is exceptionally useful for studying the molecular mechanisms of HSV reactivation, because infectious virus can be efficiently recovered upon explantation and culture of latently infected sensory ganglia (reviewed in Fraser & Valyi-Nagy, 1993). Using this model, we have shown that cellular IE factors oct-1, fos, jun and myc are induced at early times following explantation of latently infected trigeminal ganglia (TG) (Tal-Singer et al., 1997, Valyi-Nagy et al., 1991).

The present invention provides cellular factors with a putative role in the reactivation process of Herpesviruses, particularly HSV-1, HSV-2, varicella zoster virus (herein “VZV”), Epstein Barr virus (herein “EBV”) and human cytomegalovirus (herein “HCMV”). This invention was made, in part, using differential display RT-PCR (DDRT-PCR), which allows the visualization and subsequent isolation of cDNAs corresponding to mRNAs displaying altered expression in different cell populations (Liang et al., 1995, Liang & Pardee, 1992). Using this method, levels of gene expression in TG populations derived from various time points following explantation were compared. Thus, any factors modulated during the first 4 hours, the period in which viral gene expression is first detected (Devi-Rao et al., 1994, Tal-Singer et al., 1997), are believed to be important in the initial stimulation of latent viral genomes.

Certain previous studies have used DDRT-PCR to identify genes that are involved in neural stress and injury (Inokuchi et al., 1996, Kiryu et al., 1995, Qu et al., 1996). For example, Kiryu et al. (Kiryu et al., 1995) demonstrated differential expression of the rat neuronal glutamate transporter in axotomized hypoglossal motor neurons. Since glutamate transporter expression was induced in response to neuronal injury, it may be involved in the regeneration process. Others used DDRT-PCR to isolate a novel neuropeptide, called melanin-concentrating hormone, in the hypothalamic response to starvation (Qu et al., 1996). DDRT-PCR has also been used to identify cellular genes modulated by SV40 and EBV transformation (Sompayrac et al., 1996, Yan et al., 1996). However, it is a novel approach to study viral reactivation.

This invention demonstrates the isolation of forty eight differentially-displayed cDNAs representing transcripts whose levels were altered within the first 4 hours following explantation of latently infected TG. Five cDNAs were identical to murine TIS7, whose sequence has been shown to be related to interferons (IFNs) (Skup et al., 1982, Tirone & Shooter, 1989, Varnum et al., 1989). Rapid induction of IFNs α and β in neuronal cells of TG explants was also detected. In addition, other factors, such as the transcriptional activator IRF-1 (Interferon Regulatory Factor-1), and the mitogen TNF-α (tumor necrosis factor-α), were induced by explantation. The Applicants believe that HSV reactivation involves the induction of a regulatory pathway shared with IFN-related genes and herein provide certain invention embodiments based on this important discovery.

### BRIEF DESCRIPTION OF THE DRAWINGS

FIG. 1 shows PCR differential-display of cDNA derived from latently-infected mouse TG following explantation. Autoradiograph of radiolabeled DDRT-PCR products. Arrows denote PCR products representing (Left Panel) band #64 amplified with 3′ primer #2 and 5′ primer #7, (Right Panel) band #56 amplified with 3′ primer #2 and 5′ primer #3.

FIG. 2 shows sequences of DDRT-PCR products. cDNAs isolated from differential-display gels were reamplified using primers that included the T7 promoter sequence, and PCR products sequenced. Sequences were analyzed by BLAST searches. (Panel A) BLAST output using band #56 as query sequence (P value=7.7×10⁻⁴⁹) demonstrates sequence identity with murine TIS7. (Panel B) Alignment of each DDRT band with TIS7 mRNA. Five cDNA bands corresponded to mouse IFN-related gene TIS7 mRNA. Band #56 (primers:#2, #7), band #64 (primers #2,#3), band #116 (#6,#2), and band #125 (#6, #7), band #201 (#8, #2).

FIG. 3 shows confirmation of differential display. RNA was prepared from uninfected TG explants at 0, 1, 2, and 4 h p.e. Complementary DNA from differentially displayed band #56 was reamplified by PCR using 3′ primers that included the T7 promoter. PCR products were used as templates to prepare riboprobes labeled with ³²P-UTP, and added to each RNA sample. Following hybridization at 37° C. and RNase digestion, samples were separated by PAGE. (Panel A) The input probe (P) and protected fragments were visualized using phosphorimager screens. (Panel B) The intensity of each protected fragment was quantitated using Imagequant software. Fold induction was expressed as the ratio between each band to the 0 time point. (Panel C) Complementary DNA from latently-infected TG explants at 0-24 hours post explantation was subjected to PCR using primers specific for TIS7 (TIS7A set). Products were separated on 2.5% agarose gels stained with ethidium bromide, and visualized by fluorimager analysis.

FIG. 4 shows detection of IFN-β, IRF-1, IFNαβR, and IRF-2 transcripts in murine TG following explantation. RT-PCR was used to detect (Panel A) IFN-β, (Panel B) IRF-1, (Panel C) IFNαβR, (Panel D) IRF-2, and each was compared to the level of cyclophilin mRNA. Duplicate samples of TG explant RNA from 0, 1, 2, and 4 h post-explantation were analysed. Products were separated by agarose gel electrophoresis, followed by fluorimager scanning and analysis using Imagequant software. The relative amount of cDNA is expressed in these graphs in arbitrary units representing the ratio between the intensity of the PCR-product band to the intensity of cyclophilin. The ratio at the 0 time point is designated as 1. L indicates latent.

FIG. 5 shows immunostaining of interferon protein in TG following. Latently-infected or uninfected BALB/c mice were sacrificed, TG were excised, and incubated in culture media for 0-24 h p.e. Paraffin-embedded sections were processed as described in Methods and reacted with rabbit polyclonal antisera against IFN α and β. (A) 0 h p.e. of latently infected TG, (B) 4 h, (C) 8 h, (D) 24 h., (E) 0 h p.e. of uninfected TG, (F) 4 h p.e. of uninfected TG. The experiment was repeated twice, and duplicate slides were screened.

FIG. 6 shows RT-PCR detection of TNF-α, cyclophilin, and β-actin transcripts in murine TG cultured for varying times post explantation. RNA from latently-infected TG explants was prepared and analyzed by RT-PCR for (Panel A) TNF-α, and (Panel B) β-actin, and cyclophilin, as described in Materials and Methods. Products were visualized by ethidium bromide staining as shown in the inserts. The graphs represent the ratio between the PCR product band and the cyclophilin band. The ratio at the time of explantation (0) was determined as 1. Experiments were done in duplicate in four separate experiments.

FIG. 7 shows the locations and sequences of IRF-1 consensus binding sites in the genome of HSV-1, with reference to the nucleotide numbering system of GENEMBL entry HE1CG.

FIG. 8 shows that IRF-1 binds specifically to HSV-1 LAT DNA consensus sequence. In vitro translated human IRF-1 was incubated with ³²-P-labeled LAT probe in the presence or absence of antisera specific for IRF-1 or IRF-2. Samples were analyzed by electromobility shift assay (EMSA). Probe alone was used as control.

FIG. 9 shows that IRF-2 binds specifically to LAT DNA. Competition experiments were carried out with mutant LAT oligonucleotides.

FIG. 10 shows that TIS7 and IRF-1 are induced by hyperthermia measured by heat shock protein 70 (HSP70) induction. Groups of latently infected mice (n=4) were subjected to 10 minutes transient hyperthermia and sacrificed at 1, 2, 6, 15, and 24 hours post-treatment. RNA was prepared from TG and subjected to RT-PCR. Products were analyzed as described in FIG. 6. Samples were obtained from individual mice represented in each time point by colored bars Untreated mice were used as controls.

### SUMMARY OF THE INVENTION

This invention provides cellular proteins from murine trigeminal ganglia induced by the stress of injury in viral infection and/or reactivation.

In accordance with another aspect of the present invention, there are provided polynucleotides (DNA or RNA) which bind such polypeptides, or which alter the biological activity of such polypeptides.

In particular the invention provides polynucleotides having the DNA sequences given herein.

The invention also relates to novel oligonucleotides derived from the sequences given herein which can act as PCR primers in the process herein described to determine, for exaple, whether or not the genes identified herein in whole or in part are expressed in an infected tissue or individual. It is recognised that such sequences will also have utility in diagnosis of the stage of infection and type of infection the pathogen has attained. The proteins so identified are also useful as targets in screens designed to identify antiviral compounds.

In accordance with yet a further aspect of the present invention, there is provided the use of a polypeptide and/or polynucleotide of the invention for therapeutic or prophylactic purposes, for example, as an antiviral agent or a vaccine.

In accordance with another aspect of the present invention, there is provided the use of a polynucleotide and/or polypeptide of the invention for therapeutic or prophylactic purposes.

In accordance with yet another aspect of the present invention, there are provided inhibitors or activators to such polypeptides and/or polynucleotides, useful as antiviral agents. In particular, there are provided antibodies against such polypeptides and polypeptide-polynucleotide complexes.

Another aspect of the invention is a pharmaceutical composition comprising the above polypeptide, polynucleotide, inhibitor or activator of the invention and a pharmaceutically acceptable carrier.

In a particular aspect the invention provides the use of an activator or inhibitor of the invention as an antiviral agent.

The invention further relates to the manufacture of a medicament for such uses, particularly a medicament to treat HSV-1 and/or HSV-2 infection or reactivation.

Further provided, is a composition comprising IRF-1, particularly used as a screening target for the development of prophylactic and therapeutic compounds, such as compounds that interfere with HSV (herein “HSV” means HSV-1 and/or HSV-2) or VZV reactivation.

Also provided by the invention is a method for inhibiting the binding of IRF-1 to a polynucleotide, such as a DNA element (for example, ISRE's), particulary DNA comprising or near a viral origins of replication.

A further aspect of the invention is a composition comprising an IRF-1: IRF-BP complex.

A method for screening for a compound capable of interfering with HSV reactivation agonizing or antagonizing binding of IRF-1 to an inhibitor factor, such as IRF-BP.

Also provided are compounds capable of agonizing or antagonizing any compound in IRF-1 and/or interferon genetic regulatory pathway. Preferred embodiments provide such compounds to treat HSV infection and/or reactivation (herein “reactivation” means viral reactivation from latency).

The invention also provides compositions comprising IRF-1 binding site consensus sequences in HSV oriL and oriS and HSV long terminal repeats.

### GLOSSARY OF TERMS

The following definitions are provided to facilitate understanding of certain terms used frequently herein.

“Factor(s) of the invention” refers, among others, to a polypeptide comprising the amino acid sequence any or a combination of the factors involved in viral infection and/or reactivation disclosed herein, such as IRF-1, TIS7, IFN-α and IFN-β, or an allelic variant thereof; also included are polynucleotides encoding any of these polypeptides.

“Biological Activity of the Receptor” refers to the metabolic or physiologic function of said Factors of the invention including similar activities or improved activities or these activities with decreased undesirable side-effects. Also included are antigenic and immunogenic activities of said Factors of the invention.

“Factor gene(s)” refers to a polynucleotide comprising the nucleotide sequence encoding a Factor of the invention or allelic variants thereof and/or their complements.

“Antibodies” as used herein includes polyclonal and monoclonal antibodies, chimeric, single chain, and humanized antibodies, as well as Fab fragments, including the products of an Fab or other immunoglobulin expression library.

“Isolated” means altered “by the hand of man” from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide separated from the coexisting materials of its natural state is “isolated”, as the term is employed herein.

“Polynucleotide” generally refers to any polyribonucleotide or polydeoxribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as posttranslational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. See, for instance, PROTEINS—STRUCTURE AND MOLECULAR PROPERTIES, 2nd Ed., T. E. Creighton, W. H. Freeman and Company, New York, 1993 and Wold, F., Posttranslational Protein Modifications: Perspectives and Prospects, pgs. 1-12 in POSTTRANSLATIONAL COVALENT MODIFICATION OF PROTEINS, B. C. Johnson, Ed., Academic Press, New York, 1983; Seifter et al., “Analysis for protein modifications and nonprotein cofactors”, Meth Enzymol (1990) 182:626-646 and Rattan et al., “Protein Synthesis: Posttranslational Modifications and Aging”, Ann NY Acad Sci (1992) 663:48-62.

“Variant” as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis.

“Identity” is a measure of the identity of nucleotide sequences or amino acid sequences. In general, the sequences are aligned so that the highest order match is obtained. “Identity” per se has an art-recognized meaning and can be calculated using published techniques. See, e.g.: (COMPUTATIONAL MOLECULAR BIOLOGY, Lesk, A. M., ed., Oxford University Press, New York, 1988; BIOCOMPUTING: INFORMATICS AND GENOME PROJECTS, Smith, D. W., ed., Academic Press, New York, 1993; COMPUTER ANALYSIS OF SEQUENCE DATA, PART I, Griffin, A. M., and Griffin, H. G., eds., Humana Press, New Jersey, 1994; SEQUENCE ANALYSIS IN MOLECULAR BIOLOGY, von Heinje, G., Academic Press, 1987; and SEQUENCE ANALYSIS PRIMER, Gribskov, M. and Devereux, J., eds., M Stockton Press, New York, 1991). While there exist a number of methods to measure identity between two polynucleotide or polypeptide sequences, the term “identity” is well known to skilled artisans (Carillo, H., and Lipton, D., SIAM J Applied Math (1988) 48:1073). Methods commonly employed to determine identity or similarity between two sequences include, but are not limited to, those disclosed in Guide to Huge Computers, Martin J. Bishop, ed., Academic Press, San Diego, 1994, and Carillo, H., and Lipton, D., SIAM J Applied Math (1988) 48:1073. Methods to determine identity and similarity are codified in computer programs. Preferred computer program methods to determine identity and similarity between two sequences include, but are not limited to, GCG program package (Devereux, J., et al., Nucleic Acids Research (1984) 12(1):387), BLASTP, BLASTN, FASTA (Atschul, S. F. et al., J Molec Biol (1990) 215:403).

As an illustration, by a polynucleotide having a nucleotide sequence having at least, for example, 95% “identity” to a reference nucleotide sequence of SEQ ID NO: 1 is intended that the nucleotide sequence of the polynucleotide is identical to the reference sequence except that the polynucleotide sequence may include up to five point mutations per each 100 nucleotides of the reference nucleotide sequence of SEQ ID NO: 1. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5 or 3 terminal positions of the reference nucleotide sequence or anywhere between those terminal positions, interspersed either individually among nucleotides in the reference sequence or in one or more contiguous groups within the reference sequence.

Similarly, by a polypeptide having an amino acid sequence having at least, for example, 95% “identity” to a reference amino acid sequence of is intended that the amino acid sequence of the polypeptide is identical to the reference sequence except that the polypeptide sequence may include up to five amino acid alterations per each 100 amino acids of the reference amino acid sequence. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

“Individual(s)” means a eukaryote, particularly a mammal, and especially a human, particularly one infected with a virus or believed to be infected by a virus.

“Plasmids” are designated by a lower case p preceded and/or followed by capital letters and/or numbers. The starting plasmids herein are either commercially available, publicly available on an unrestricted basis, or can be constructed from available plasmids in accord with published procedures. In addition, equivalent plasmids to those described are known in the art and will be apparent to the ordinarily skilled artisan.

“Digestion” of DNA refers to catalytic cleavage of the DNA with a restriction enzyme that acts only at certain sequences in the DNA. The various restriction enzymes used herein are commercially available and their reaction conditions, cofactors and other requirements were used as would be known to the ordinarily skilled artisan. For analytical purposes, typically 1 μg of plasmid or DNA fragment is used with about 2 units of enzyme in about 20 μl of buffer solution. For the purpose of isolating DNA fragments for plasmid construction, typically 5 to 50 μg of DNA are digested with 20 to 250 units of enzyme in a larger volume. Appropriate buffers and substrate amounts for particular restriction enzymes are specified by the manufacturer. Incubation times of about 1 hour at 37 C are ordinarily used, but may vary in accordance with the supplier's instructions. After digestion the reaction is electrophoresed directly on a polyacrylamide gel to isolate the desired fragment.

Size separation of the cleaved fragments is performed using 8 percent polyacrylamide gel described by Goeddel, D. et al., (1980) Nucleic Acids Res., 8:4057.

“Oligonucleotides” refers to either a single stranded polydeoxynucleotide or two complementary polydeoxynucleotide strands which may be chemically synthesized. Such synthetic oligonucleotides have no 5′ phosphate and thus will not ligate to another oligonucleotide without adding a phosphate with an ATP in the presence of a kinase. A synthetic oligonucleotide will ligate to a fragment that has not been dephosphorylated.

“Ligation” refers to the process of forming phosphodiester bonds between two double stranded nucleic acid fragments (Maniatis, T., et al., supra., p. 146). Unless otherwise provided, ligation may be accomplished using known buffers and conditions with 10 units to T4 DNA ligase (“ligase”) per 0.5 μg of approximately equimolar amounts of the DNA fragments to be ligated.

A “replicon” is any genetic element (e.g., plasmid, chromosome, virus) that functions as an autonomous unit of DNA replication in vivo; i.e., capable of replication under its own control.

A “vector” is a replicon, such as a plasmid, phage, or cosmid, to which another DNA segment may be attached so as to bring about the replication of the attached segment.

A “double-stranded DNA molecule” refers to the polymeric form of deoxyribonucleotides (bases adenine, guanine, thymine, or cytosine) in a double-stranded helix, both relaxed and supercoiled. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having the sequence homologous to the mRNA).

A DNA “coding sequence of” or a “nucleotide sequence encoding” a particular protein, is a DNA sequence which is transcribed and translated into a polypeptide when placed under the control of appropriate regulatory sequences.

A “promoter sequence” is a DNA regulatory region capable of binding RNA polymerase in a cell and initiating transcription of a downstream (3′ direction) coding sequence. For purposes of defining the present invention, the promoter sequence is bound at the 3′ terminus by a translation start codon (e.g., ATG) of a coding sequence and extends upstream (5′ direction) to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

DNA “control sequences” refers collectively to promoter sequences, ribosome binding sites, polyadenylation signals, transcription termination sequences, upstream regulatory domains, enhancers, and the like, which collectively provide for the expression (i.e., the transcription and translation) of a coding sequence in a host cell.

A control sequence “directs the expression” of a coding sequence in a cell when RNA polymerase will bind the promoter sequence and transcribe the coding sequence into mRNA, which is then translated into the polypeptide encoded by the coding sequence.

A “host cell” is a cell which has been transformed or transfected, or is capable of transformation or transfection by an exogenous DNA sequence.

A cell has been “transformed” by exogenous DNA when such exogenous DNA has been introduced inside the cell membrane. Exogenous DNA may or may not be integrated (covalently linked) into chromosomal DNA making up the genome of the cell. In prokaryotes and yeasts, for example, the exogenous DNA may be maintained on an episomal element, such as a plasmid. With respect to eukaryotic cells, a stably transformed or transfected cell is one in which the exogenous DNA has become integrated into the chromosome so that it is inherited by daughter cells through chromosome replication. This stability is demonstrated by the ability of the eukaryotic cell to establish cell lines or clones comprised of a population of daughter cell containing the exogenous DNA.

A “clone” is a population of cells derived from a single cell or common ancestor by mitosis. A “cell line” is a clone of a primary cell that is capable of stable growth in vitro for many generations.

A “heterologous” region of a DNA construct is an identifiable segment of DNA within or attached to another DNA molecule that is not found in association with the other molecule in nature.

### DETAILED DESCRIPTION OF THE INVENTION

Applicants have discovered that cellular interferon-related genes are induced in tissues in response to certain stimuli as described herein. The tissue studied is primarily nerve tissue (ganglia) where HSV and other Herpesviruses establish latent infections. These stimuli also induce reactivation of HSV and other Herpesviruses from latency into active replication. Applicants also discovered that IRF-1, an important cellular regulatory protein, is induced by the same stimuli, and connected this regulatory factor to HSV and other Herpesviruses replication through the identification of IRF-1 binding sites in the DNA sequence of HSV and other Herpesviruses DNA.

It is believed that IRF-1, and/or the IRF-1 regulatory pathway, are essential for reactivation of HSV and other Herpesviruses from latency, and that pharmacological interference with this pathway will interfere with HSV and other Herpesvirus reactivation resulting in arrest of clinical disease.

In view of this the invention provides various targets for screening for therapeutic compounds using the methods described herein. Such targets include, for example, IRF-1. A compound interfering with HSV and other Herpesvirus reactivation by inhibiting the binding of IRF-1 to viral DNA elements (ISRE's) may be found using such screens and are useful in antiviral therapy, especially against HSV and other Herpesviruses.

Another target, an IRF-1: IRF-BP complex, comprising IRF-1 and IRF-BP, can be used to screen for compounds interfering with HSV reactivation by, among other modes of action, freezing or mimicking the binding of IRF-1 to it's inhibitor factor IRF-BP. Such compounds are useful in antiviral therapy, especially against HSV.

Other target include any element in the IRF-1 and interferon genetic regulatory pathway which subsequently impacts HSV reactivation from latency. These compounds are useful to screen for antiviral compounds. Such compounds are useful in antiviral therapy, especially against HSV.

Yet another target are polynucleotides derived from or comrpising an IRF-1 binding site consensus sequences in HSV LAT/IPC0, oriL and oriS play a significant role in HSV replication. A preferred embodiment of this sequence is set forth in SEQ ID NO:1. Such compounds are useful in antiviral therpy, especially against HSV.

To demonstrate this reactivation, murine trigeminal ganglia explants by differential display RT-PCR (DDRT) were analyzed. Five of the DDRT hits mapped to murine TIS7, an interferon-related cellular Immediate Early gene which is activated in several cell types in response to stress (Guardavaccaro et al., 1995, Herschman et al., 1994, Tirone & Shooter, 1989, Varnum et al., 1989, Varnum et al., 1994). Using RT-PCR and in-situ hybridization it was shown that other IFN-related gene transcripts, including IFN-β, and-the interferon regulatory factor-1 (IRF-1) were induced following explantation of murine trigeminal ganglia (Tal-Singer et al. 1998).

IRF-1 is a transcriptional activator and tumor supressor protein which regulates interferon genes transcription such as IL-1 beta (via Interleukin Converting Enzyme (ICE) pathways) (Tamura et al., 1996), and MHC-1 expression (Drew et al., 1995, Hobart et al., 1997). It is activated by interferons and the STAT1 pathway (Pine et al., 1994). It binds to cellular proteins such as TFIIB, and NFκB (Drew et al., 1995, Wang et al., 1996). Its DNA binding and transcriptional activation functions are inhibited when it is bound to IRF-BP protein (Kondo, 1996). Its known functions involve the inflammatory response and regulation of the cell cycle via induction of p21 (Tanaka et al., 1996).

Other target include any element in the IRF-1 and interferon genetic regulatory pathway which subsequently impacts HSV reactivation from latency. These compounds are useful to screen for antiviral compounds. Such compounds are useful in antiviral therapy, especially against HSV.

Yet another target are polynucleotides derived from or comrpising an IRF-1 binding site consensus sequences in HSV LAT/IPC0, oriL and oriS which play a significant role in HSV replication. A preferred embodiment of this sequence is set forth in SEQ ID NO:1. Such compounds are useful in antiviral therpy, especially against HSV.

To demonstrate this reactivation, murine trigeminal ganglia explants were analyzed by differential display RT-PCR (DDRT). Five of the DDRT hits mapped to murine TIS7, an interferon-related cellular Immediate Early gene which is activated in several cell types in response to stress (Guardavaccaro et al., 1995, Herschman et al., 1994, Tirone & Shooter, 1989, Varnum et al., 1989, Varnum et al., 1994). Using RT-PCR and immunostaining it was shown that other IFN-related gene transcripts, including IFN-beta, and the interferon regulatory factor-1 (IRF-1) were induced following explantation of murine trigeminal ganglia.

IRF-1 is a transcriptional activator and tumor supressor protein which regulates interferon genes transcription such as IL-1 beta (via Interleukin Converting Enzyme (ICE) pathways) (Tamura et al., 1996), and MHC-I expression (Drew et al., 1995, Hobart et al., 1997)]. It is activated by interferons and the STAT1 pathway (Pine et at., 1994). It binds to cellular proteins such as TFIIB, and NFkB (Drew et al., 1995, Wang et al., 1996). Its DNA binding and transcriptional activation functions are inhibited when it is bound to IRF-BP protein (Kondo, 1996). Its known functions involve the inflammatory response and regulation of the cell cycle via induction of p21 (Tanaka et al., 1996).

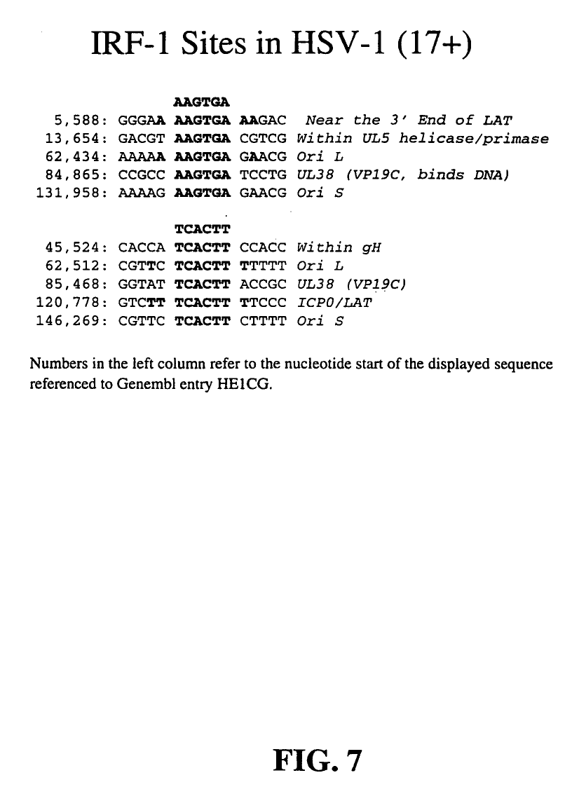

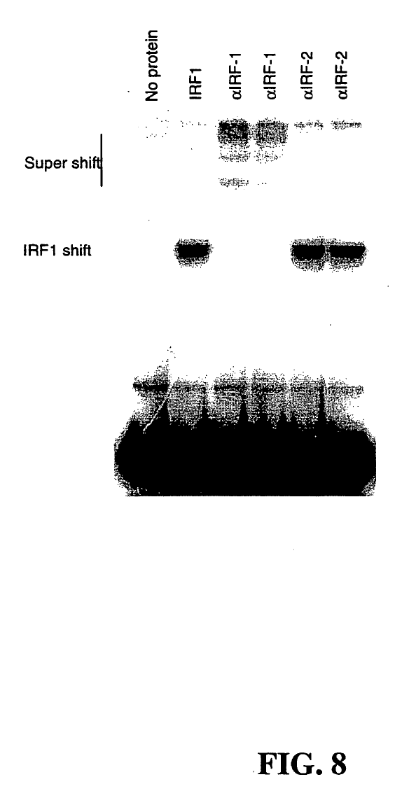

IRF-1 is a member of a multigene family which recognizes the same promoter consensus sequence (herein referred to as “ISRE” or “ICS”). Sequence analysis using Findpatterns function of the GCG software (Genetics Computer Group, Madison, Wis.). HSV-1 complete genome sequence (Genbank locus HE1CG accession # X14112) was searched for the core consensus IRF-1 site in interferon promoters using the hexamer AAGTGA (Tanaka & Taniguchi, 1992). Sequence matches are listed in Table 1 by sequence location, and gene name. Ten identical matches were found in in HSV-1 strain 17+ sequence (see FIG. 7). A strong consensus identical to sequences in IFN promoters was present in the latency associated transcript (LAT) genome location which overlaps with the ICP0 transcript. Both LAT and ICP0 are important for reactivation from latency. The applicants found that in vitro translated IRF-1 and IRF-2 can bind probes derived from this region specifically (FIGS. 8, 9). Two weaker matches mapped to the palindrome in oriL, and one mapped to oriS. These regions matched to origin binding protein (OBP or UL9) Binding Site III, which are conserved in HSV-1 and HSV-2 strains, as shown in the sequence below [SEQ ID NO:1].

```
5′ AAAAAAAGTGAGAACGCGAAGCGTTCGCACTTTGTCCTAATAATATA-
                SITE III
      TATATTATTAGGACAAAGTGCGAACGCTTCGCGTTCTCACTTTTTTT3′
                                                                  SITE III
```

These consensus sites are within regions previously identified as origin binding protein (OBP or UL9) Binding Site III. Also, of great interest are previous observations that, in addition to binding OBP, Binding Site III interacts with yet unknown cellular proteins (Dabrowski et al., 1991; Dabrowski et al., 1994). Taken together, these observations suggest that IRF-1 may bind to regulatory elements in the viral genome such as the origins of replication, and ICP0/LAT, and thereby upregulate viral replication or gene expression. Interestingly, functional IRF binding sites recently were identified in the herpesvirus Epstein-Barr virus (EBV) (Schaffer et al., 1997; Nonkwelo et al., 1997). IRF-1 and IRF-2 bind directly to consensus sequence sites in the EBV Type I latency promoter of EBNA-1 (Qp), and IRF factors may play a primary role in transcriptional regulation of EBNA-1 in cell culture (Schaffer et al., 1997; Nonkwelo et al., 1997).

Mobility shift assays using probes specific for this region in oriS and oriL (Dabrowski et al., 1994, Dabrowski & Schaffer, 1991), indicated that certain cellular proteins bind to this region. A single base mutation in this consensus sequence affected the rate of DNA replication at late times following infection of cultured cells. We have been unable to show that in-vitro translated human IRF-1 can bind probes from this region. However, it does not rule out the possibility that IRF-1 bound to other viral or cellular factors can bind at the origin of replication in its latent conformation.

Further sequence analysis of other Herpesviruses genomes revealed other IRF-1 consensus sequences. These consensus sequences provided by the invention include, for example, the following sequences in Table 1. Highly conserved nucleotides surrounding the core are bold and underlined.

TABLE 1

IRF-1 consensus sites in HSV-1 Strain 17+ by
Pattern Searching using the AAGTGA IRF-1 Core
consensus

| | |
|---|---|
| 5,588: GGGAA AAGTGA AAGAC | Near the 3′ End of LAT |
| 13,654: GACGT AAGTGA CGTCG | In UL5 helicase/primase |
| 62,434: AAAAA AAGTGA_GAACG | Ori L |
| 84,865: CCGCC_AAGTGA TCCTG | UL38 (VP19C, binds DNA) |
| 131,958: AAAAG AAGTGA GAACG | Ori S |

TCACTT IRF-1 rev Core consensus

| | |
|---|---|
| 45,524: CACCA TCACTT CCACC | Within gH |
| 62,512: CGTTC TCACTT TTTTT | Ori L |
| 85,468: GGTAT TCACTT ACCGC | UL38 (VP19C) |
| 120,778: GTCTT_TCACTT TTCCC | ICP0/LAT |
| 146,269: CGTTC TCACTT CTTTT | Ori S |

Human Cytomegalovirus: Hehcmvcg.Gb_vi
1 AAGTGA

| | |
|---|---|
| 35,131: GCCGA AAGTGA AAGTA | |
| 52,719: GAAAG AAGTGA AACCC | |
| 53,842: CGCGC AAGTGA AGCCG | |
| 56,326: GTTAA AAGTGA TTTTT | |
| 59,418: CAATA AAGTGA GTCTA | |
| 81,191: CAAAT AAGTGA TAATG | |
| 95,257: AAAAG AAGTGA TACAA | Auxilliary replication |

region 94860-95670

121,335: GGCTA AAGTGA CAGGA
128,147: CTTCG AAGTGA ATATT
155,734: GCAGA AAGTGA TGTGG
158,557: ATCCT AAGTGA GGTGA
158,891: CTACA AAGTGA AGAAT
163,229: AGCCT AAGTGA CGGTG

1 /Rev TCACTT

```
  15,215: GTGTG TCACTT GTTGC
  15,695: CAACT TCACTT CAAAC
  16,798: AAGAT TCACTT AAAGC
  50,301: TATAA TCACTT TCCGC
  70,132: ACGTA TCACTT TCACG
  87,029: GGCCA TCACTT CGGGG
  97,452: TCGCA TCACTT AAGAA
130,301: ATCGA TCACTT TTTTC
139,862: CGACG TCACTT TGAGC
157,414: AAAGT TCACTT ATTCT
169,360: GTTTT TCACTT ACTGA
171,562: AGAAC TCACTT AAGAG
174,668: AAAAA TCACTT TTGGA
175,038: GTCGT TCACTT TGCCG
184,562: GTTAA TCACTT TAAGT
209,216: GGATC TCACTT ACCGC
224,280: GCCGT TCACTT TTCCG
```

Varicella Zoster Virus: Hevzvxx.Gb_Vi
1 AAGTGA

| | |
|---|---|
| 3,179: TCCAA AAGTGA CTTCG | |
| 5,514: AAGAC AAGTGA ACCCT | |
| 9,053: GCTGA AAGTGA AGTGG | ORF7 |
| 15,581: AAGGA AAGTGA GACCG | Upsteram of ORF12 |
| 25,015: CATAC AAGTGA CAACC | |
| 27,340: ATGCC AAGTGA TCGGT | |
| 27,744: GGTTG AAGTGA ATGCC | |
| 36,842: ATTTT AAGTGA TCCAA | |
| 38,369: ACGTT AAGTGA ACTTA | |
| 42,071: TATCA AAGTGA TTTTA | Upstream of ORF 23 |
| 47,587: CTGTT AAGTGA_GCCAA | |
| 58,021: TTTAT AAGTGA_AACAA | ORF31 |
| 71,172: TTGCA AAGTGA GGTTA | |
| 87,744: ACCCA AAGTGA ACATC | End of ORF50 |
| 104,738: CAGCT AAGTGA CATTT | |
| 115,777: GTTTT AAGTGA CTATA | |
| 121,897: GTGGG AAGTGA AACTA | ORF 71 Downstrem of |

ORI 119547-119810
1 /Rev TCACTT

| | |
|---|---|
| 4,271: ACTTT TCACTT TCCCC | |
| 43,348: TAATG TCACTT TGCAT | |
| 44,942: AGTTG TCACTT TTAGC | |
| 67,385: AACTT TCACTT CTTTT | |
| 70,980: CTACA TCACTT TCGAC | |
| 73,891: GCCCT TCACTT TGTGG | |
| 75,664: GAAGT TCACTT AGCCC | |
| 82,708: TTCAT TCACTT TGGTC | Promoter for ORF46 |
| 97,452: AGACA TCACTT ACGTG | |
| 101,928: TATAA TCACTT TTCTT | |
| 104,274: TTGTA TCACTT ACGAT | |
| 112,936: TCGGA TCACTT TATAA | Unknown region |

Upstream of ORF66
Epstein Barr Virus: Ebv.Gb_Vi
1 AAGTGA

| | |
|---|---|
| 6,198: AAGCA AAGTGA AGGGC | Upstream of ORI P the |

latency origin (7315-9312) EBRI promoter
(PolIII transcript associated with SNRPs

| | |
|---|---|
| 17,640: GACCG AAGTGA AGGCC | Bam HI W/W' repeats |

near TATAAAG for EBNA LP
(latent protein)

| | |
|---|---|
| 20,712: GACCG AAGTGA AGGCC | |
| 23,784: GACCG AAGTGA AGGCC | |
| 26,856: GACCG AAGTGA AGGCC | |
| 29,928: GACCG AAGTGA AGGCC | |
| 33,000: GACCG AAGTGA AGGCC | |
| 36,072: GACCG AAGTGA AGGCC | |
| 39,144: GACCG AAGTGA AGGCC | |
| 42,216: GACCG AAGTGA AGGCC | |
| 45,288: GACCG AAGTGA AGGCC | End of Bam HI W/W' |
| repeat | |
| 58,005: TGTAA AAGTGA AGCTG | 924 bp downstream of |
| BFL2 promoter TATAAAG | |
| 74,427: CTGGA AAGTGA CTCGG | |
| 81,082: CAAGA AAGTGA ACCAG | 250 bp downstream of |
| BMRF2 | |
| 93,389: GAAGA AAGTGA GGACA | |
| 149,877: AGCAC AAGTGA TTAGG | |

1 /Rev TCACTT

```
        179: CCCTC TCACTT CTACT
        267: GGTTG TCACTT GTGAG
        335: ACAGT TCACTT CCTCT
    5,558: ACCCC TCACTT TGTAC
    9,637: ATAAA TCACTT CCCTA
  11,783: CGGGG TCACTT CCCCT
  11,845: TGGTG TCACTT CCGCA
  53,582: ATGCA TCACTT TGAGC
  97,786: GACCA TCACTT AAGTT
107,114: GATAA TCACTT TTATC
150,593: TAGGA TCACTT TCATA
```

In view of Applicants findings provided herein, Applicants believe that members of the IRF-1 family are involved in regulation of HSV and other Herpesviruses replication or transcription in host cells.

Applicants have also used, for example, DDRT-PCR to identify genes differentially expressed following the stress caused by explantation of latently-infected murine TG. Mice were infected by the corneal route with HSV-1, and at 4 weeks post infection, mice were sacrificed and the TG were explanted. RNA was prepared from TG at various times following explantation, followed by DDRT-PCR.

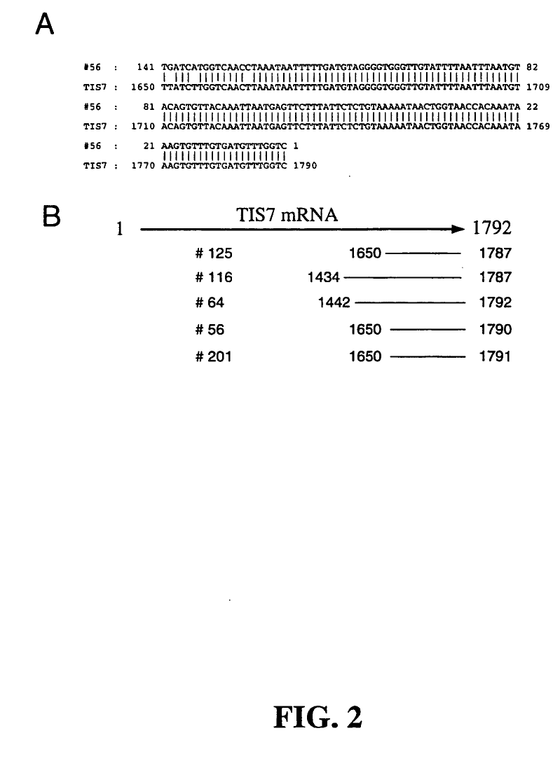

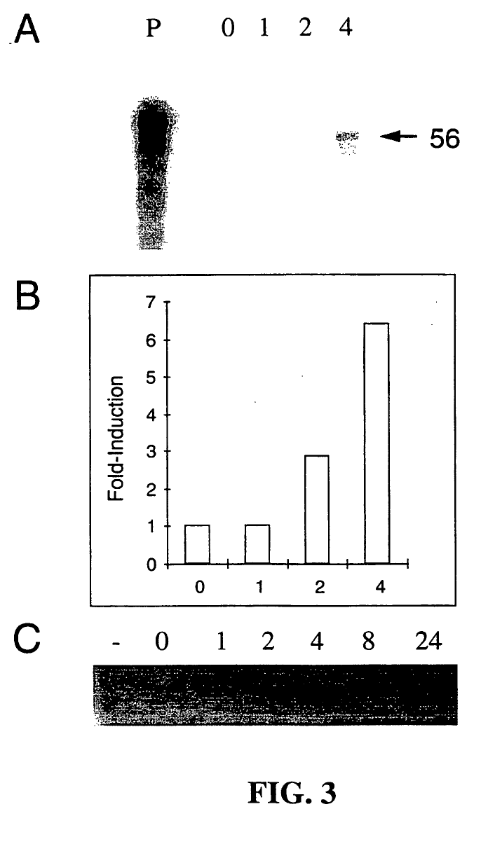

TIS7 is induced by the stress of explantation. Five differentially displayed bands were identified as overlapping regions of murine TIS7 (Lim et al., 1987, Varnum et al., 1989) (FIG. 2) and the results were confirmed by quantitative RPA and RT-PCR (FIG. 3). The TIS (TPA-inducible sequences) family members are early response genes (Walz et al., 1976) which are induced rapidly and transiently in Swiss 3T3 cells by the tumor promoter mitogen tetradecanoyl phorbol acetate (TPA) (Lim et al., 1987), or by serum (Herschman, 1991). Most of the TIS genes also have been identified in rat PC12 cells, following induction with NGF, TPA, epidermal growth factor and depolarization (Kujubu et al., 1987). Moreover, TIS induction has been detected in primary astrocyte cultures following mitogen induction (Arenalder et al., 1989). Thus, a family of TIS genes appear to constitute a common pathway or response to many cell stimulatory agents or physical stimuli.

The pattern of induction previously observed for TIS7/PC4 genes in these systems revealed an increase in the levels of RNA or protein between 2-4 hours post stimulus (Varnum et al., 1994), similar to our observation in the mouse explant model (FIG. 2). Moreover, Applicants have shown that another TIS transcript, TIS28 or c-fos, was induced rapidly following explantation (Tal-Singer et al., 1997, Valyi-Nagy et al., 1991), and again, the kinetics match those previously observed following mitogen induction (Arenalder et al., 1989). These observations suggest that explantation and mitogen stimulation induce similar cellular early response pathways which either activate or induce TIS genes. Applicants believed that these pathways also may be among the earliest events involved in the induction of latent herpesvirus and demonstarted this in the present invention. It has been reported that rat PC4 (highly related to murine TIS7) and c-fos are both induced by activation of the oncogenenic protein-tyrosine kinase v-fps, encoded in the retrovirus Fujinami sarcoma virus (Jahner & Hunter, 1991). It would be of interest to determine whether a similar kinase, either of viral or cellular origin, is activated in explanted ganglia or in other HSV reactivation systems.

Interferon β is induced by the stress of explantation. TIS7 was originally identified as a gene induced in murine 3T3 cells following infection with Newcastle disease virus (Skup et al., 1982). Nucleotide sequence analysis revealed some conservation with human IFN-β and rat IFN-γ (Tirone & Shooter, 1989). The present invention provides that, like TIS7, IFN-α and IFN-β are induced in neuronal cells within the first hour following explantation. These observations indicate that these interferon related genes, although different in function, share a common cellular pathway that is putatively involved in the early events of HSV reactivation.

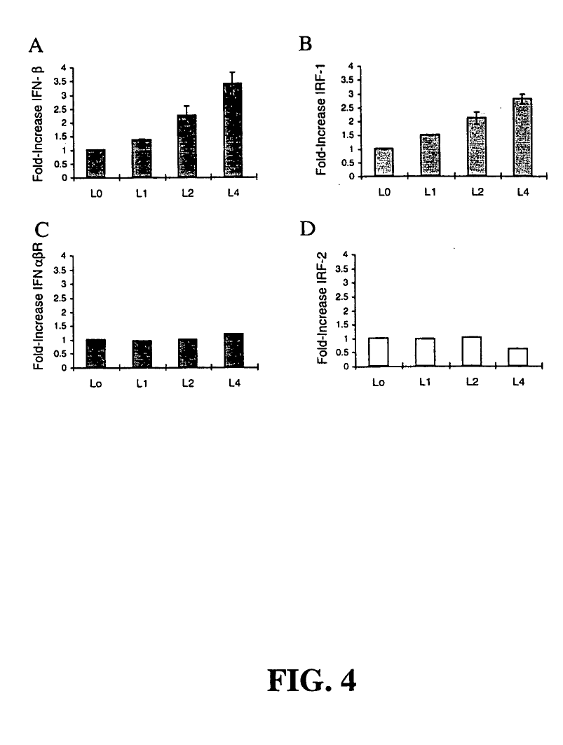

IFN-β expression is modulated at the transcriptional level by multiple regulatory factors that bind upstream of the initiation site, such as the activators IRF-1 and NFkB, and the repressor IRF-2 (reviewed in Tanaka & Taniguchi, 1992). Applicants showed that IRF-1 was induced by the stress of explantation. In contrast, neither IRF-2 nor the IFN receptor were induced (FIG. 4). Since the induction of IRF-1 followed the same temporal pattern as that of IFN, IFN-alpha and beta are believed to be induced via an IRF-1 dependent pathway in explanted TG cells (Kawakami et al., 1995). Induction of IFN expression has previously been observed following HSV infection (Gobi et al., 1988b, Green et al., 1991, Stanwick et al., 1982a). However, Applicants detected induction of IFN at 1-2 hours, prior to viral gene induction, which was detected at 2-4 h p.e. (Tal-Singer et al., 1997). Furthermore, IFN induction was detected in neurons of both latently-infected and uninfected TG. IFN-b and IRF-1 were induced in the TG in the absence of serum in the explantation media. Taken together, these data indicate that IFN induction is a consequence of the stress of explantation, and did not result from viral gene expression, or from incubation in the presence of serum factors.

It has been suggested that, during HSV infection, transcription of IFN-b is induced by complex formation between cellular Oct-1 and viral VP16, and the interaction of this complex with Oct-1 binding sites in the IFN promoter (Leblanc & Hiscott, 1992). Consistent with this, Applicants have shown previously that Oct-1 is induced in neuronal cells within 1 hour p.e. (Tal-Singer et al., 1997, Valyi-Nagy et al., 1991). However, IFN induction occurred even in the absence of latent virus. Thus, Applicants believe that cellular factors that are functionally analogous to the viral late gene and virion tegument component VP16 are induced in TG explants. A precedent for this is the identification of a cellular Oct-1-binding protein in lymphocytes (called either Bob-1, OCAB, or OCBP), that appears to be required for the tissue-specific induction of immunoglobulin gene promoters (Gstaiger et al., 1995, Luo & Roeder, 1995, Strubin et al., 1995). Thus, Applicants believe that neuronal-specific Oct-1 binding proteins are induced in explanted ganglia. This gene-activation pathway could be shared by IFN and viral promoters since both contain Oct-1 binding consensus sequences (MacDonald et al., 1990, O'Hare & Goding, 1988).

Relationship between reactivating HSV and IFNs. IFN is known to possess antiviral properties, and indeed is induced by HSV infection (Gobl et al., 1988a, Green et al., 1981, Stanwick et al., 1982b). Several mechanisms have been elucidated for specific effects of IFN on HSV. For example, IFN is an inhibitor of activation of HSV immediate early (IE) genes in vitro by VP16 (LaMarco & McKnight, 1989). Applicants recently found induction of both Early and IE HSV genes during the first hours of viral reactivation (Tal-Singer et al., 1997). The absence of viral IE gene expression prior to Early gene expression during the first hours following explantation of TG may be a result of the inhibitory effect of IFN on a neuron-specific VP16 homologue.

Furthermore, IFN blocks both HSV morphogenesis and release of viral particles from infected cells (Chatterjee et al., 1985). Therefore, an interesting relationship between IFN and reactivating HSV may involve one round of viral replication, allowing the virus to travel from TG neurons to the site of recrudescence in corneal epithelium. Immediate induction of IFN may prevent the spread of reactivating virus within the nervous system by inhibiting release of viral particles and activating host defense mechanisms such as natural killer cells (Habu et al., 1984). Moreover, in another virus system, neuroblastoma cells expressing high levels of IFN-b support persistent rabies virus infections (Honda et al., 1985). This suggests that the IFN response may be involved in ensuring the viability of infected host cells.

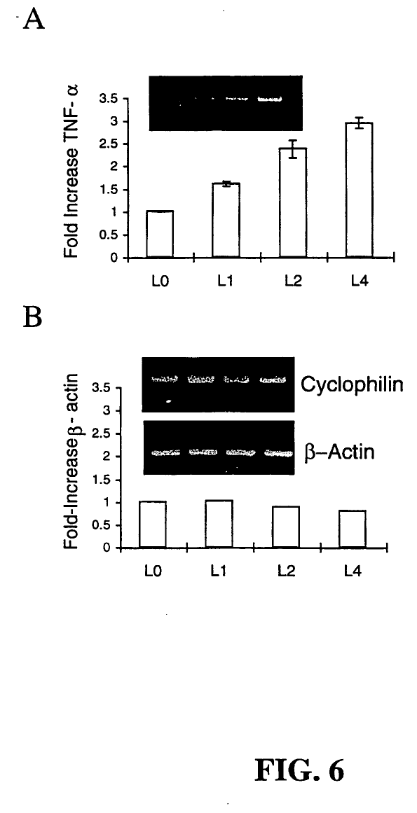

In a different scenario, IFN may inhibit viral reactivation in neurons and, in a few cells, other cellular factors override its effects by inducing high levels of viral gene expression. For example, Walev et al. (Walev et al., 1995) have shown that treatment of TG explants with soluble IFN inhibits reactivation, detected by reduction of infectious virus in the presence of IFN. In contrast, TNF-a treatment induced the efficiency of reactivation in that study. These data support the hypothesis that induction of IFN inhibits multiple rounds of viral replication in neuronal cells of the TG. Consistent with this scenario, we detected induction of TNF-a transcription in TG explants (FIG. 6) under conditions leading to viral reactivation. Thus, it is possible that the levels of TNF-a in few neurons (1% of latent neurons) are higher than the levels of IFN, allowing the latent viral genome in those neurons to reactivate.

The relationship of TIS7, IFN, and viral responses. TIS7 and interferons are induced by viral infection (Skup et al., 1982, Tanaka & Taniguchi, 1992) and, as shown in this study, by the stress of explantation. The precise function of TIS7 in the cell is yet to be elucidated, it is however clear that it plays a role in cellular growth and differentiation (Guardavaccaro et al., 1995, Herschman et al., 1994). Furthermore, it is known that TIS7 has no antiviral activity (Tirone & Shooter, 1989) which is a property of interferons (Tanaka & Taniguchi, 1992). Applicants performed studies to determine whether these cellular components share a common induction pathway with HSV. Applicnts believe that that TIS, interferon family members, and HSV share a common regulatory element such as IRF-1, and that HSV-1 has evolved reactivation strategies which take advantage of these cellular stress-induced activation pathways.

Using DDRT-PCR several genes involved in the cellular response to the stress of explantation were identified. These results also yielded insights into the cellular environment which is present during HSV reactivation. Therefore, PCR differential-display represents an excellent method to screen for species of RNA which are transcriptionally regulated in TG following explantation. Genes identified using this method can be studied in other HSV reactivation systems, resulting in a database of specific genes which may be involved in the reactivation process.

Also described by the invention are differential display results showing that TIS7 was found in 5 separate bands, while only one other band was found more than one time, thus prompting the focus on TIS7.

TIS7 induction was shown in neurons at the protein level, using immunohistochemistry of TG sections.

Provided herein, to better describe the invention, are putative IRF-1 binding in the viral genome, FIG. 7 (there were significant matches to the IRF-1 consensus binding site). The disclosure of these sites in no way limits the manner in which cellular factors interact with virus sequences during reactivation. Applicants have also demonstrated that IRF-1 binding sites occur in potentially critical regions of the viral genome, and thus are believed to be crucial in the reactivation process.

The mechanisms provided herein by which the Factors of the invention function in viral infection and latency in no way limit the scope of the invention. These mechanisms are provided to clarify the invention and certain bases for the invention.

Identified herein are cellular genes induced in response to stimuli that reactivate virus and methods for screening compounds to treat disease based on this observation. Therapies based on this observation are also provided. The invention provides that these genes are potential causative agents in reactivation, but this model in no way limit the scope of the invention.

Certain of the polynucleotides of the invention, such as IRF-1, TIS7, interfereon alpha, and interfereon beta, are well known in the art, and references disclosing their sequences are provided herein. Each of the references provided herein are incorporated by reference herein in their entirety.

Any polynucleotide of the present invention may be in the form of RNA or in the form of DNA, which DNA includes cDNA, genomic DNA, and synthetic DNA. The DNA may be double-stranded or single-stranded, and if single stranded may be the coding strand or non-coding (anti-sense) strand. The coding sequence which encodes the polypeptide may be identical to the coding sequence shown or may be a different coding sequence which coding sequence, as a result of the redundancy or degeneracy of the genetic code, encoding the same polypeptide.

The present invention includes variants of the hereinabove described polynucleotides which encode fragments, analogues and derivatives of the polypeptide characterized by the deduced amino acid sequence given herein. The variant of the polynucleotide may be a naturally occurring allelic variant of the polynucleotide or a non-naturally occurring variant of the polynucleotide.

Thus, the present invention includes polynucleotides encoding the same polypeptide characterized by the deduced amino acid sequence given herein as well as variants of such polynucleotides which variants encode for a fragment, derivative or analogue of the polypeptide. Such nucleotide variants include deletion variants, substitution variants and addition or insertion variants.

The polynucleotide may have a coding sequence which is a naturally occurring allelic variant of the coding sequence characterized by the DNA sequence disclosed herein. As known in the art, an allelic variant is an alternate form of a polynucleotide sequence which may have a substitution, deletion or addition of one or more nucleotides, which does not substantially alter the function of the encoded polypeptide.

The polynucleotide which encodes for the mature polypeptide, may include only the coding sequence for the mature polypeptide or the coding sequence for the mature polypeptide and additional coding sequence such as a leader or secretory sequence or a proprotein sequence.

Thus, the term “polynucleotide encoding a polypeptide” encompasses a polynucleotide which includes only coding sequence for the polypeptide as well as a polynucleotide which includes additional coding and/or non-coding sequence.

The present invention therefore includes polynucleotides, wherein the coding sequence for the mature polypeptide may be fused in the same reading frame to a polynucleotide sequence which aids in expression and secretion of a polypeptide from a host cell, for example, a leader sequence which functions as a secretory sequence for controlling transport of a polypeptide from the cell. The polypeptide having a leader sequence is a preprotein and may have the leader sequence cleaved by the host cell to form the mature form of the polypeptide. The polynucleotides may also encode for a proprotein which is the mature protein plus additional 5′ amino acid residues. A mature protein having a prosequence is a proprotein and may be an inactive form of the protein. Once the prosequence is cleaved an active mature protein remains.

Thus, for example, the polynucleotide of the present invention may code for a mature protein, or for a protein having a prosequence or for a protein having both a prosequence and a presequence (leader sequence). Further, the amino acid sequences provided herein show a methionine residue at the NH₂-terminus. It is appreciated, however, that during post-translational modification of the peptide, this residue may be deleted. Accordingly, this invention contemplates the use of both the methionine-containing and the methionineless amino terminal variants of each protein disclosed herein.

The polynucleotides of the present invention may also have the coding sequence fused in frame to a marker sequence at either the 5′ or 3′ terminus of the gene which allows for purification of the polypeptide of the present invention. The marker sequence may be a hexa-histidine tag supplied by the pQE series of vectors (supplied commercially by Quiagen Inc.) to provide for purification of the polypeptide fused to the marker in the case of a bacterial host.

The present invention further relates to polynucleotides which hybridize to the hereinabove-described sequences if there is at least 50% and preferably at least 70% identity between the sequences. The present invention particularly relates to Streptococcal polynucleotides which hybridize under stringent conditions to the hereinabove-described polynucleotides . As herein used, the term “stringent conditions” means hybridization will occur only if there is at least 95% and preferably at least 97% identity between the sequences. The polynucleotides which hybridize to the hereinabove described polynucleotides in a preferred embodiment encode polypeptides which retain substantially the same biological function or activity as the polypeptide characterised by the deduced amino acid sequence given herein.

The terms “fragment,” “derivative” and “analogue” when referring to the polypeptide characterized by the deduced amino acid sequence herein, means a polypeptide which retains essentially the same biological function or activity as such polypeptide. Thus, an analogue includes a proprotein which can be activated by cleavage of the proprotein portion to produce an active mature polypeptide.

The polypeptide of the present invention may be a recombinant polypeptide, a natural polypeptide or a synthetic polypeptide, preferably a recombinant polypeptide.

The fragment, derivative or analogue of the polypeptide characterized by the deduced amino acid sequence herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the polypeptide is fused with another compound, such as a compound to increase the half-life of the polypeptide (for example, polyethylene glycol), or (iv) one in which the additional amino acids are fused to the polypeptide, such as a leader or secretory sequence or a sequence which is employed for purification of the polypeptide or a proprotein sequence. Such fragments, derivatives and analogues are deemed to be within the scope of those skilled in the art from the teachings herein.

The polypeptides and polynucleotides of the present invention are preferably provided in an isolated form, and preferably are purified to homogeneity.

The term “isolated” means that the material is removed from its original environment (e.g., the natural environment if it is naturally occurring). For example, a naturally-occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment.

The present invention also relates to vectors which include polynucleotides of the present invention, host cells which are genetically engineered with vectors of the invention and the production of polypeptides of the invention by recombinant techniques.

In accordance with yet a further aspect of the present invention, there is therefore provided a process for producing the polypeptide of the invention by recombinant techniques by expressing a polynucleotide encoding said polypeptide in a host and recovering the expressed product. Alternatively, the polypeptides of the invention can be synthetically produced by conventional peptide synthesizers.

Host cells are genetically engineered (transduced or transformed or transfected) with the vectors of this invention which may be, for example, a cloning vector or an expression vector. The vector may be, for example, in the form of a plasmid, a cosmid, a phage, etc. The engineered host cells can be cultured in conventional nutrient media modified as appropriate for activating promoters, selecting transformants or amplifying the genes. The culture conditions, such as temperature, pH and the like, are those previously used with the host cell selected for expression, and will be apparent to the ordinarily skilled artisan.

Suitable expression vectors include chromosomal, nonchromosomal and synthetic DNA sequences, e.g., bacterial plasmids; phage DNA; baculovirus; yeast plasmids; vectors derived from combinations of plasmids and phage DNA. However, any other vector may be used as long as it is replicable and viable in the host.

The appropriate DNA sequence may be inserted into the vector by a variety of procedures. In general, the DNA sequence is inserted into an appropriate restriction endonuclease site(s) by procedures known in the art.

The DNA sequence in the expression vector is operatively linked to an appropriate expression control sequence(s) (promoter) to direct mRNA synthesis. As representative examples of such promoters, there may be mentioned: LTR or SV40 promoter, the E. coli. lac or trp, the phage lambda P_{L} promoter and other promoters known to control expression of genes in eukaryotic or prokaryotic cells or their viruses. The expression vector also contains a ribosome binding site for translation initiation and a transcription terminator. The vector may also include appropriate sequences for amplifying expression.

In addition, the expression vectors preferably contain one or more selectable marker genes to provide a phenotypic trait for selection of transformed host cells such as dihydrofolate reductase or neomycin resistance for eukaryotic cell culture, or such as tetracycline or ampicillin resistance in E. coli.

The gene can be placed under the control of a promoter, ribosome binding site (for bacterial expression) and, optionally, an operator (collectively referred to herein as “control” elements), so that the DNA sequence encoding the desired protein is transcribed into RNA in the host cell transformed by a vector containing this expression construction. The coding sequence may or may not contain a signal peptide or leader sequence. The polypeptides of the present invention can be expressed using, for example, the E. coli tac promoter or the protein A gene (spa) promoter and signal sequence. Leader sequences can be removed by the bacterial host in post-translational processing. See, e.g., U.S. Pat. Nos. 4,431,739; 4,425,437; 4,338,397. Promoter regions can be selected from any desired gene using CAT (chloramphenicol transferase) vectors or other vectors with selectable markers. Two appropriate vectors are PKK232-8 and PCM7. Particular named bacterial promoters include lacd, lacZ, T3, T7, gpt, lambda P_{R}, P_{L} and trp. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art.

In addition to control sequences, it may be desirable to add regulatory sequences which allow for regulation of the expression of the protein sequences relative to the growth of the host cell. Regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences.

An expression vector is constructed so that the particular coding sequence is located in the vector with the appropriate regulatory sequences, the positioning and orientation of the coding sequence with respect to the control sequences being such that the coding sequence is transcribed under the “control” of the control sequences (i.e., RNA polymerase which binds to the DNA molecule at the control sequences transcribes the coding sequence). Modification of the coding sequences may be desirable to achieve this end. For example, in some cases it may be necessary to modify the sequence so that it may be attached to the control sequences with the appropriate orientation; i.e., to maintain the reading frame. The control sequences and other regulatory sequences may be ligated to the coding sequence prior to insertion into a vector, such as the cloning vectors described above. Alternatively, the coding sequence can be cloned directly into an expression vector which already contains the control sequences and an appropriate restriction site.

Generally, recombinant expression vectors will include origins of replication and selectable markers permitting transformation of the host cell, e.g., the ampicillin resistance gene of E. coli and S. cerevisiae TRP1 gene, and a promoter derived from a highly-expressed gene to direct transcription of a downstream structural sequence. The heterologous structural sequence is assembled in appropriate phase with translation initiation and termination sequences, and preferably, a leader sequence capable of directing secretion of translated protein into the periplasmic space or extracellular medium. Optionally, the heterologous sequence can encode a fusion protein including an N-terminal identification peptide imparting desired characteristics, e.g., stabilization or simplified purification of expressed recombinant product.

The vector containing the appropriate DNA sequence as hereinabove described, as well as an appropriate promoter or control sequence, may be employed to transform an appropriate host to permit the host to express the protein.

More particularly, the present invention also includes recombinant constructs comprising one or more of the sequences as broadly described above. The constructs comprise a vector, such as a plasmid or viral vector, into which a sequence of the invention has been inserted, in a forward or reverse orientation. In a preferred aspect of this embodiment, the construct further comprises regulatory sequences, including, for example, a promoter, operably linked to the sequence. Large numbers of suitable vectors and promoters are known to those of skill in the art, and are commercially available. The following vectors are provided by way of example. Bacterial: pET-3 vectors (Stratagene), pQE70, pQE60, pQE-9 (Qiagen), pbs, pD10, phagescript, psiX174, pbluescript SK, pbsks, pNH8A, pNH16a, pNH18A, pNH46A (Stratagene); ptrc99a, pKK223-3, pKK233-3, pDR540, pRIT5 (Pharmacia). Eukaryotic: pBlueBacIII (Invitrogen), pWLNEO, pSV2CAT, pOG44, pXT1, pSG (Stratagene) pSVK3, pBPV, pMSG, pSVL (Pharmacia). However, any other plasmid or vector may be used as long as they are replicable and viable in the host.

Examples of recombinant DNA vectors for cloning and host cells which they can transform include the bacteriophage 1 (E. coli), pBR322 (E. coli), pACYC177 (E. coli), pKT230 (gram-negative bacteria), pGV1106 (gram-negative bacteria), pLAFR1 (gram-negative bacteria), pME290 (non-E. coli gram-negative bacteria), pHV14 (E. coli and Bacillus subtilis), pBD9 (Bacillus), pIJ61 (Streptomyces), pUC6 (Streptomyces), YIp5 (Saccharomyces), a baculovirus insect cell system, YCp19 (Saccharomyces). See, generally, “DNA Cloning”: Vols. I & II, Glover et al. ed. IRL Press Oxford (1985) (1987) and; T. Maniatis et al. (“Molecular Cloning” Cold Spring Harbor Laboratory (1982).

In some cases, it may be desirable to add sequences which cause the secretion of the polypeptide from the host organism, with subsequent cleavage of the secretory signal.

Polypeptides can be expressed in host cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook, et al., Molecular Cloning: A Laboratory Manual, Second Edition, Cold Spring Harbor, N.Y., (1989), the disclosure of which is hereby incorporated by reference.

Following transformation of a suitable host strain and growth of the host strain to an appropriate cell density, the selected promoter is induced by appropriate means (e.g., temperature shift or chemical induction) and cells are cultured for an additional period.

Cells are typically harvested by centrifugation, disrupted by physical or chemical means, and the resulting crude extract retained for further purification.

Microbial cells employed in expression of proteins can be disrupted by any convenient method, including freeze-thaw cycling, sonication, mechanical disruption, or use of cell lysing agents, such methods are well known to those skilled in the art.

Depending on the expression system and host selected, the polypeptide of the present invention may be produced by growing host cells transformed by an expression vector described above under conditions whereby the polypeptide of interest is expressed. The polypeptide is then isolated from the host cells and purified. If the expression system secretes the polypeptide into growth media, the polypeptide can be purified directly from the media. If the polypeptide is not secreted, it is isolated from cell lysates or recovered from the cell membrane fraction. Where the polypeptide is localized to the cell surface, whole cells or isolated membranes can be used as an assayable source of the desired gene product. Polypeptide expressed in bacterial hosts such as E. coli may require isolation from inclusion bodies and refolding. Where the mature protein has a very hydrophobic region which leads to an insoluble product of overexpression, it may be desirable to express a truncated protein in which the hydrophobic region has been deleted. The selection of the appropriate growth conditions and recovery methods are within the skill of the art.

The polypeptide can be recovered and purified from recombinant cell cultures by methods including ammonium sulphate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the mature protein. Finally, high performance liquid chromatography (HPLC) can be employed for final purification steps.

Depending upon the host employed in a recombinant production procedure, the polypeptides of the present invention may be glycosylated or may be non-glycosylated. Polypeptides of the invention may also include an initial methionine amino acid residue.

The polypeptide may be used as an antigen for vaccination of a host to produce specific antibodies which have anti-bacterial action. This invention also contemplates the use of the DNA encoding the antigen as a component in a DNA vaccine as discussed more fully below.

The polypeptides or cells expressing them can be used as an immunogen to produce antibodies thereto. These antibodies can be, for example, polyclonal or monoclonal antibodies. The term antibodies also includes chimeric, single chain, and humanized antibodies, as well as Fab fragments, or the product of an Fab expression library. Various procedures known in the art may be used for the production of such antibodies and fragments.

Antibodies generated against the polypeptides of the present invention can be obtained by direct injection of the polypeptides into an animal or by administering the polypeptides to an animal, preferably a nonhuman. The antibody so obtained will then bind the polypeptides itself. In this manner, even a sequence encoding only a fragment of the polypeptides can be used to generate antibodies binding the whole native polypeptides. Such antibodies can then be used to isolate the polypeptide from tissue expressing that polypeptide.

Polypeptide derivatives include antigenically or immunologically equivalent derivatives which form a particular aspect of this invention.

The term ‘antigenically equivalent derivative’ as used herein encompasses a polypeptide or its equivalent which will be specifically recognised by certain antibodies which, when raised to the protein or polypeptide according to the present invention, interfere with the interaction between pathogen and mammalian host.

The term ‘immunologically equivalent derivative’ as used herein encompasses a peptide or its equivalent which when used in a suitable formulation to raise antibodies in a vertebrate, the antibodies act to interfere with the interaction between pathogen and mammalian host.

In particular derivatives which are slightly longer or slightly shorter than the native protein or polypeptide fragment of the present invention may be used. In addition, polypeptides in which one or more of the amino acid residues are modified may be used. Such peptides may, for example, be prepared by substitution, addition, or rearrangement of amino acids or by chemical modification thereof. All such substitutions and modifications are generally well known to those skilled in the art of peptide chemistry.

The polypeptide, such as an antigenically or immunologically equivalent derivative or a fusion protein thereof is used as an antigen to immunize a mouse or other animal such as a rat or chicken. The fusion protein may provide stability to the polypeptide. The antigen may be associated, for example by conjugation, with an immunogenic carrier protein for example bovine serum albumin (BSA) or keyhole limpet haemocyanin (KLH). Alternatively a multiple antigenic peptide comprising multiple copies of the protein or polypeptide, or an antigenically or immunologically equivalent polypeptide thereof may be sufficiently antigenic to improve immunogenicity so as to obviate the use of a carrier.

For preparation of monoclonal antibodies, any technique which provides antibodies produced by continuous cell line cultures can be used. Examples include the hybridoma technique (Kohler and Milstein, Nature, 256:495-497(1975)), the trioma technique, the human B-cell hybridoma technique (Kozbor et al., Immunology Today 4:72(1983)), and the EBV-hybridoma technique to produce human monoclonal antibodies (Cole, et al., 1985, in Monoclonal Antibodies and Cancer Therapy, Alan R. Liss, Inc., pp. 77-96).

Techniques described for the production of single chain antibodies (U.S. Pat. No. 4,946,778) can be adapted to produce single chain antibodies to immunogenic polypeptide products of this invention.

Using the procedure of Kohler and Milstein (supra, (1975)), antibody-containing cells from the immunised mammal are fused with myeloma cells to create hybridoma cells secreting monoclonal antibodies.

The hybridomas are screened to select a cell line with high binding affinity and favorable cross reaction with other Streptococcal species using one or more of the original polypeptide and/or the fusion protein. The selected cell line is cultured to obtain the desired Mab.

Hybridoma cell lines secreting the monoclonal antibody are another aspect of this invention.

Alternatively phage display technology could be utilised to select antibody genes with binding activities towards the polypeptide either from repertoires of PCR amplified v-genes of lymphocytes from humans screened for possessing anti-Fbp or from naive libraries (McCafferty, J. et al., Nature 348:552-554(1990), and Marks, J. et al., Biotechnology 10:779-783(1992)). The affinity of these antibodies can also be improved by chain shuffling (Clackson, T. et al., Nature 352:624-628(1991)).

The antibody should be screened again for high affinity to the polypeptide and/or fusion protein.

As mentioned above, a fragment of the final antibody may be prepared.

The antibody may be either intact antibody of Mᵣ approx 150,000 or a derivative of it, for example a Fab fragment or a Fv fragment as described in Skerra, A and Pluckthun, A., Science 240:1038-1040 (1988). If two antigen binding domains are present each domain may be directed against a different epitope—termed ‘bispecific’ antibodies.

The antibody of the invention may be prepared by conventional means for example by established monoclonal antibody technology (Kohler, G. and Milstein, C. (supra, (1975) or using recombinant means e.g. combinatorial libraries, for example as described in Huse, W. D. et al., Science 246:1275-1281 (1989).

Preferably the antibody is prepared by expression of a DNA polymer encoding said antibody in an appropriate expression system such as described above for the expression of polypeptides of the invention. The choice of vector for the expression system will be determined in part by the host, which may be a prokaryotic cell, such as E. coli (preferably strain B) or Streptomyces sp. or a eukaryotic cell, such as a mouse C127, mouse myeloma, human HeLa, Chinese hamster ovary, filamentous or unicellular fungi or insect cell. The host may also be a transgenic animal or a transgenic plant (for example, as described in Hiatt, A. et al., Nature 340:76-78(1989). Suitable vectors include plasmids, bacteriophages, cosmids and recombinant viruses, derived from, for example, baculoviruses and vaccinia.

The Fab fragment may also be prepared from its parent monoclonal antibody by enzyme treatment, for example using papain to cleave the Fab portion from the Fc portion.

Preferably the antibody or derivative thereof is modified to make it less immunogenic in the patient. For example, if the patient is human the antibody may most preferably be ‘humanised’; where the complimentarity determining region(s) of the hybridoma-derived antibody has been transplanted into a human monoclonal antibody, for example as described in Jones, P. et al., Nature 321:522-525 (1986), or Tempest et al., Biotechnology 9:266-273 (1991).

The modification need not be restricted to one of ‘humanisation’; other primate sequences (for example Newman, R. et al., Biotechnology 10:1455-1460 (1992)) may also be used.

The humanised monoclonal antibody, or its fragment having binding activity, form a particular aspect of this invention.

This invention provides a method of screening drugs to identify those which interfere with the proteins selected as targets herein, which method comprises measuring the interference of the activity of the protein by a test drug. For example if the protein selected has a catalytic activity, after suitable purification and formulation the activity of the enzyme can be followed by its ability to convert its natural substrates. By incorporating different chemically synthesised test compounds or natural products into such an assay of enzymatic activity one is able to detect those additives which compete with the natural substrate or otherwise inhibit enzymatic activity.

The invention also relates to inhibitors identified thereby.

The use of a polynucleotide of the invention in genetic immunisation will preferably employ a suitable delivery method such as direct injection of plasmid DNA into muscles (Wolff et al., Hum. Mol. Genet. 1:363 (1992); Manthorpe et al., Hum. Gene Ther. 4:419 (1963)), delivery of DNA complexed with specific protein carriers ( Wu et al, J. Biol. Chem. 264:16985 (1989)), coprecipitation of DNA with calcium phosphate (Benvenisty & Reshef, Proc. Nat'l Acad. Sci. USA, 83:9551 (1986)), encapsulation of DNA in various forms of liposomes (Kaneda et al., Science 243:375 (1989)), particle bombardment (Tang et al., Nature 356:152 (1992)); Eisenbraun et al., DNA Cell Biol. 12:791 (1993)) and in vivo infection using cloned retroviral vectors (Seeger et al., Proc. Nat'l. Acad. Sci. USA 81:5849 (1984)). Suitable promoters for muscle transfection include CMV, RSV, SRa, actin, MCK, alpha globin, adenovirus and dihydrofolate reductase.

In therapy or as a prophylactic, the active agent i.e., the polypeptide, polynucleotide or inhibitor of the invention, may be administered to a patient as an injectable composition, for example as a sterile aqueous dispersion, preferably isotonic.

Alternatively the composition may be formulated for topical application for example in the form of ointments, creams. lotions, eye ointments, eye drops, ear drops, mouthwash, impregnated dressings and sutures and aerosols, and may contain appropriate conventional additives, including, for example, preservatives, solvents to assist drug penetration, and emollients in ointments and creams. Such topical formulations may also contain compatible conventional carriers, for example cream or ointment bases, and ethanol or oleyl alcohol for lotions. Such carriers may constitute from about 1% to about 98% by weight of the formulation; more usually they will constitute up to about 80% by weight of the formulation.

For administration to human patients, it is expected that the daily dosage level of the active agent will be from 0.01 to 10 mg/kg, typically around 1 mg/kg. The physician in any event will determine the actual dosage which will be most suitable for an individual patient and will vary with the age, weight and response of the particular patient. The above dosages are exemplary of the average case. There can, of course, be individual instances where higher or lower dosage ranges are merited, and such are within the scope of this invention.

A vaccine composition is conveniently in injectable form. Conventional adjuvants may be employed to enhance the immune response.

A suitable unit dose for vaccination is 0.5-5 ug/kg of antigen, and such dose is preferably administered 1-3 times and with an interval of 1-3 weeks.

Within the indicated dosage range, no adverse toxicologicals effects are expected with the compounds of the invention which would preclude their administration to suitable patients.

Screening Assays

A Factor of the invention may be employed in a screening process for compounds which bind the receptor and which activate (agonists) or inhibit activation of (antagonists) the receptor polypeptide of the present invention. Thus, polypeptides of the invention may also be used to assess the binding of small molecule substrates and ligands in, for example, cells, cell-free preparations, chemical libraries, and natural product mixtures. These substrates and ligands may be natural substrates and ligands or may be structural or functional mimetics. See Coligan et al., Current Protocols in Immunology 1(2):Chapter 5 (1991).

Factors of the invention are responsible for many biological functions, including many pathologies. Accordingly, it is desirous to find compounds and drugs which stimulate a Factor of the invention on the one hand and which can inhibit the function of a Factor of the invention on the other hand. In general, agonists are employed for therapeutic and prophylactic purposes for such conditions as Herpesvirus infections, such as HSV, VZV, HCMV and EBV infections. Antagonists may be employed for a variety of therapeutic and prophylactic purposes for such conditions as Herpesvirus infection, such as HSV, VZV, HCMV and EBV infections.

In general, such screening procedures involve producing appropriate cells which express the receptor polypeptide of the present invention on the surface thereof. Such cells include cells from mammals, yeast, Drosophila or E. coli. Cells expressing the receptor (or cell membrane containing the expressed receptor) are then contacted with a test compound to observe binding, or stimulation or inhibition of a functional response.

The assays may simply test binding of a candidate compound wherein adherence to the cells bearing the receptor is detected by means of a label directly or indirectly associated with the candidate compound or in an assay involving competition with a labeled competitor. Further, these assays may test whether the candidate compound results in a signal generated by activation of the receptor, using detection systems appropriate to the cells bearing the receptor at their surfaces. Inhibitors of activation are generally assayed in the presence of a known agonist and the effect on activation by the agonist by the presence of the candidate compound is observed.

cDNA, protein and antibodies to a Factor of the invention may also be used to configure assays for detecting the effect of added compounds on the production of mRNA and protein from a Factor of the invention in cells. For example, an ELISA may be constructed for measuring secreted or cell associated levels of protein feom a Factor of the invention using monoclonal and polyclonal antibodies by standard methods known in the art, and this can be used to discover agents which may inhibit or enhance the production of a Factor of the invention (also called antagonist or agonist, respectively) from suitably manipulated cells or tissues. Standard methods for conducting screening assays are well understood in the art.

Examples of potential antagonists of a Factor of the invention include antibodies or, in some cases, oligonucleotides or proteins which are closely related to the ligand of a Factor of the invention, e.g., a fragment of the ligand, or small molecules which bind to the receptor but do not elicit a response, so that the activity of the receptor is prevented.

Prophylactic and Therapeutic Methods

This invention provides methods of treating abnormal conditions related to both an excess of and insufficient amounts of the biological activity of a Factor of the invention.

If the activity of a Factor of the invention is in excess, several approaches are available. One approach comprises administering to a subject an inhibitor compound (antagonist) as hereinabove described along with a pharmaceutically acceptable carrier in an amount effective to inhibit activation by blocking binding of ligands to a Factor of the invention, or by inhibiting a second signal, and thereby alleviating the abnormal condition.

In another approach, soluble forms of a Factor of the invention polypeptides still capable of binding the ligand in competition with endogenous Factor of the invention may be administered. Typical embodiments of such competitors comprise fragments of the polypeptide from a Factor of the invention.

In still another approach, expression of the gene encoding endogenous Factor of the invention can be inhibited using expression blocking techniques. Known such techniques involve the use of antisense sequences, either internally generated or separately administered. See, for example, O'Connor, J Neurochem (1991) 56:560 in Oligodeoxynucleotides as Antisense Inhibitors of Gene Expression, CRC Press, Boca Raton, Fla. (1988). Alternatively, oligonucleotides which form triple helices with the gene can be supplied. See, for example, Lee et al., Nucleic Acids Res (1979) 6:3073; Cooney et al., Science (1988) 241:456; Dervan et al., Science (1991) 251:1360. These oligomers can be administered per se or the relevant oligomers can be expressed in vivo.

For treating abnormal conditions related to an under-expression of Factor of the invention and its activity, several approaches are also available. One approach comprises administering to a subject a therapeutically effective amount of a compound which activates Factor of the invention, i.e., an agonist as described above, in combination with a pharmaceutically acceptable carrier, to thereby alleviate the abnormal condition. Alternatively, gene therapy may be employed to effect the endogenous production of a Factor of the invention by the relevant cells in the subject. For example, a polynucleotide of the invention may be engineered for expression in a replication defective retroviral vector, as discussed above. The retroviral expression construct may then be isolated and introduced into a packaging cell transduced with a retroviral plasmid vector containing RNA encoding a polypeptide of the present invention such that the packaging cell now produces infectious viral particles containing the gene of interest. These producer cells may be administered to a subject for engineering cells in vivo and expression of the polypeptide in vivo. For overview of gene therapy, see Chapter 20, Gene Therapy and other Molecular Genetic-based Therapeutic Approaches, (and references cited therein) in Human Molecular Genetics, T Strachan and A P Read, BIOS Scientific Publishers Ltd (1996).

Diagnostic Assays

This invention also relates to the use of a Factor of the invention polynucleotides for use as diagnostic reagents. Detection of a differentially expressed Factor gene associated with a viral infection and/or reactivation, as compared to a normal individual, will provide a diagnostic tool that can add to or define a diagnosis of a disease or susceptibility to a disease which results from under-expression, over-expression or altered expression of a Factor gene.

Nucleic acids for diagnosis may be obtained from a subject's cells, such as from blood, urine, saliva, tissue biopsy or autopsy material. The genomic DNA may be used directly for detection or may be amplified enzymatically by using PCR or other amplification techniques prior to analysis. RNA or cDNA may also be used in similar fashion. Deletions and insertions can be detected by a change in size of the amplified product in comparison to the normal genotype. Point mutations can be identified by hybridizing amplified DNA to labeled Factor gene-derived nucleotide sequences. Perfectly matched sequences can be distinguished from mismatched duplexes by RNase digestion or by differences in melting temperatures. DNA sequence differences may also be detected by alterations in electrophoretic mobility of DNA fragments in gels, with or without denaturing agents, or by direct DNA sequencing. See, e.g., Myers et al., Science (1985) 230:1242. Sequence changes at specific locations may also be revealed by nuclease protection assays, such as RNase and S1 protection or the chemical cleavage method. See Cotton et al., Proc Natl Acad Sci USA (1985) 85: 4397-4401. In another embodiment, an array of oligonucleotides probes comprising Factor gene nucleotide sequence or fragments thereof can be constructed to conduct efficient screening of e.g., genetic mutations. Array technology methods are well known and have general applicability and can be used to address a variety of questions in molecular genetics including gene expression, genetic linkage, and genetic variability. (See for example: M. Chee et al.. Science, Vol 274, pp 610-613 (1996)).

The diagnostic assays offer a process for diagnosing or determining a susceptibility to Herpesvirus infection, such as HSV, VZV, HCMV and EBV infection, through detection of mutation or difference in expression in the Factor of gene as compared to normal individuals by the methods described.

In addition, Herpesvirus infection, such as HSV, VZV, HCMV and EBV infection, can be diagnosed by methods comprising determining from a sample derived from a subject an abnormally decreased or increased level of a polypeptide or mRNA or a Factor of the invention. Decreased or increased expression can be measured at the RNA level using any of the methods well known in the art for the quantitation of polynucleotides, such as, for example, PCR, RT-PCR, RNase protection, Northern blotting and other hybridization methods. Assay techniques that can be used to determine levels of a protein, such as an a Factor of the invention, in a sample derived from a host are well-known to those of skill in the art. Such assay methods include radioimmunoassays, competitive-binding assays, Western Blot analysis and ELISA assays.

The examples below are carried out using standard techniques, which are well known and routine to those of skill in the art, except where otherwise described in detail. The examples illustrate, but do not limit the invention.

### EXAMPLES

### Example 1

### Infection of Mice and Reactivation Experiments

4 to 6 week old female BALB/c BYJ mice were obtained from Jackson Laboratories. Mice were anesthetized with intraperitoneal injection of ketamine (87 mg/kg)/xylazine (13 mg/kg), then, after corneal scarification, were inoculated in the eye with 10⁴ PFU of HSV-1 17⁺ (Brown et al., 1973). At a minimum of 28 days post infection, mice were sacrificed by cervical dislocation and TG were isolated. Groups of 6-10 explanted TG were incubated in Dulbecco's Modified Eagle medium supplemented with 5% fetal bovine serum at 37° C. for 0, 1, 2, 4 or 24 hours post-explant (p.e.). In a single experiment, TG were explanted in the absence of serum. For hyperthermia experiments, mice were infected following light scarification of both ear pinnae with 10⁵ PFU of HSV-1 SC-16 (Harbour et al. 1981). At a minimum of 28 days post infection, transient hyperthermia was induced as described previously (Sawtell et al. 1992). Briefly, mice were placed in a 43° C. water bath for 10-12 minutes, then dried and placed in a warm incubator (34° C.) for approximately 30 min to prevent hypothermia. At varying times post treatment, mice were sacrificed by cervical dislocation and TG were removed.

### Example 2

### Extraction of RNA

Ganglia used for RNA preparation were snap frozen in liquid nitrogen. RNA was isolated from TG and brain stems using the TRIzol reagent, as described by manufacturer (Gibco BRL), followed by extensive digestion with RNase-free DNase I (BMB) and ethanol precipitation. RNA concentrations were determined by spectrophotometer and agarose gel electrophoresis (Maniatis et al., 1982).

### Example 3

### Differential Display RT-PCR

Complementary DNA (cDNA) was prepared from 300 ng RNA from latently-infected TG at 0, 1, 2, and 4 hours p.e. using the Differential Display Kit™ (Display Systems Biotech, Inc., Los Angeles, Calif.), as described by manufacturer. Primers presented in this study are listed in Table 2. Briefly, RNA from each sample was incubated, with one of nine downstream primers containing 11T residues and 2 nucleotide anchors (AA, AC, AG, CA, CC, CG, GA, GC, GG), for one hour at 40° C., followed by 5 min at 95° C. to inactivate the M-MuLV enzyme. cDNA was stored at −70° C. Each cDNA was subjected to PCR amplification with DisplayTaq™ (Display Systems Biotech) using the original downstream primer, one of 24 10-mer 5′ primers, and a-³²P-dATP (Warthoe, 1995). PCR conditions were 35 cycles of 30 sec denaturation at 94° C., 60 sec primer annealing at 40° C., and 60 sec extension at 72° C., employing a Perkin Elmer Cetus Gene Amp PCR System thermocycler. A final extension reaction was then performed for 5 min 72° C. Radiolabeled reaction products were subjected to high resolution polyacrylamide/urea gel electrophoresis as described (Liang & Pardee, 1992). Gels were dried on Whatman filters and analyzed by autoradiography. Differentially-displayed PCR bands were cut out from the filter paper and dissolved in DEPC-treated water (Ambion) for 30 min at room temperature followed by 10 min at 100° C.

TABLE 2

Differential Display primers presented in
this study*.

| | |
|---|---|
| Name | Sequence |

| | |
|---|---|
| 5′ #1 | GATCATAGCC |
| 5′ #2 | CTGCTTGATG |
| 5′ #3 | GATCCAGTAC |
| 5′ #4 | GATCGCATTG |
| 5′ #5 | AAACTCCGTC |
| 5′ #6 | TGGTAAAGGG |
| 5′ #7 | GATCATGGTC |
| 5′#9 | GTTTTCGCAG |
| 5′#10 | TACCTAAGCG |
| 5′#11 | GATGTGACAC |
| 5′#12 | GATCTAACCG |
| 5′#13 | TGGATTGGTC |
| 5′#14 | GGAACCAATC |
| 5′#15 | GATCAATCGC |
| 5′#20 | GATCAAGTCC |
| 5′#21 | GATCTCAGAC |
| 5′#22 | GGTACTAAGG |
| 3′ # 2 | TTTTTTTTTTTAC |
| 3′ #4 | TTTTTTTTTTTCA |
| 3′ #5 | TTTTTTTTTTTCC |
| 3′ #6 | TTTTTTTTTTTCG |
| 3′ #8 | TTTTTTTTTTTGC |

*Primers obtained from Display Systems Biotech, Inc., Los Angeles, CA

### Example 4

### Reamplification PCR

To assure that each band analyzed contained a single cDNA species, each differentially displayed band was reamplified in 4 individual PCR reactions. Four 17-T11VVN 3′ primers were used, where VV was the original DDRT nucleotide anchor, T7 was a 23 nucleotide portion of the T7 promoter (TAATACGACTC ACTATAGGGCCC), and N was A,G,T, or C. The Reamplification reactions included the original upstream primers, 2 mM dNTP and the Stoffel fragment of Taq Polymerase (Perkin Elmer Cetus). Reactions were performed under the original DDRT-PCR conditions. PCR products were separated by agarose electrophoresis and the most prominent product among the four parallel reactions was isolated for automated sequencing and further confirmation.

Sequence analysis using these primers showed differntially displayed bands as outlined in Table 2 below.

TABLE 2

| | | | | | GenBank | |
|---|---|---|---|---|---|---|
| Band | 3′ | 5′ | Expression | | Accession | |
| Number | Primer | Primer | Pattern^{a} | cDNA Name | Number | P Value^{b} |
| 64^{c} | 2 | 3 | Ind | Mouse TIS7 | V00756 | 1e−210 |
| 56^{c} | 2 | 7 | Ind | Mouse TIS7 | J00424 | 1e−75 |
| 116^{c} | 6 | 2 | Ind | Mouse TIS7 | X17400 | 1e−140 |
| 125^{c} | 6 | 7 | Ind | Mouse TIS7 | X17400 | 1e−37 |
| 201^{c} | 8 | 2 | Ind | Mouse TIS7 | X17400 | 1e−47 |
| 114^{d} | 6 | 1 | Rep | Unknown mouse | W97484 | 1e−66 |
| 117^{d} | 6 | 2 | Ind | Human DNA Binding Protein | D28468 | 1e−21 |
| 200 | 6 | 1 | Rep | Unknown human | W38244 | 1e−70 |
| 229 | 8 | 12 | Rep | Mouse Semaphorin | X85990 | 1e−189 |
| 115^{d} | 6 | 1 | Ind | Unknown human | R61599 | 1e−36 |
| 124^{d} | 6 | 7 | Rep | Unknown human | R61599 | 1e−29 |
| 39 | 2 | 1 | Ind | Unknown human | D19792 | 1e−18 |
| 42 | 2 | 2 | Ind | Unknown human | D62695 | 1e−66 |
| 44 | 2 | 2 | Ind | Unknown human | T34888 | 1e−45 |
| 65 | 2 | 4 | Rep | Mouse kallikrein tumor antigen | M18620 | 1e−13 |
| 97 | 5 | 15 | Ind | Mouse T-cell Antigen 4-1BB | U02567 | 1e−52 |
| 27 | 4 | 9 | Ind | Rat ATPase | H39388 | 1e−22 |
| 123 | 6 | 5 | Ind | GAS5 mouse growth arrest gene | X67267 | 1e−45 |
| 20 | 4 | 20 | Rep | Human NADH ubiquinone oxyreductase | T58895 | 1e−185 |
| | | | | subunit B14 | | |
| 129 | 6 | 11 | Ind | Human nuclear encoded mitochondrial | M25484 | 1e−168 |
| | | | | NADH ubiquinone oxyreductase 24 KD | | |
| | | | | subunit | | |
| 232 | 8 | 12 | Ind | Mouse laminin | T54408 | 1e−116 |
| 41 | 2 | 2 | Rep | Mouse alpha-tubulin | H34265 | 1e−73 |
| 54 | 2 | 6 | Ind | Mouse retroransposon-like element | M21123 | 1e−66 |
| 138 | 6 | 15 | Ind | Mouse beta-tubulin | X04663 | 1e−38 |
| 98 | 5 | 15 | Rep | Mouse beta-tubulin | X04663 | 1e−32 |
| 21 | 4 | 21 | Rep | Human ribosomal protein S26 | X77770 | 0 |
| 218 | 8 | 6 | Rep | Mouse lecithin cholesterolacyl transferase | X54095 | 1e−15 |
| 53 | 2 | 6 | Ind | Human retrovirus-related reverse | K02590 | 1e−153 |
| | | | | transcriptase | | |

Key to Table 2:
^{a}Ind = induced, Rep = repressed
^{b}Represents Poisson distribution value. The cutoff number representing significant hits was 1e−9
^{c}Bands 56, 64, 116, 125, and 201 share sequences (see FIG. 2)
^{d}Bands pairs 114/117 and 115/124 share overlapping sequences and database hits.

### Example 5

### Confirmation of Differentially Regulated Expression

Isolated reamplification bands were used as templates for synthesis of ³²P labeled riboprobes with the MAXIscript™ kit (Ambion., Austin, Tex.) as described by manufacturer. RNase protection assays (RPA) were performed using Hybspeed™ RPA kit (Ambion), and 0.5-1 mg total RNA from a second, new set of latently-infected and uninfected TG explants. Mouse β-actin riboprobes provided with the MAXIscript™ kit were used as controls. Probes and protected fragments were analyzed by denaturing polyacrylamide gel electrophoresis (PAGE) and phosphorimaging.

### Example 6

### PCR Amplification of cDNA

The second confirmatory PCR using specific primer sets was performed as follows. cDNA was generated from 2 mg of total RNA using Superscript Preamplification kit priming with oligo (dT) and random hexamers (Gibco BRL). Reactions were performed in 25-ml volumes containing 4% of cDNA, 200 mM each deoxynucleoside triphosphate (Pharmacia), 1 mM of each primer, 1.25 U of AmpliTaq Gold™ (Perkin Elmer) with PCR Buffer A (Fisher). Primer pairs used are described in Table 3

TABLE 3
PCR Primer Pairs Used in This Study

| | | | |
|---|---|---|---|
| | | Product | |
| Name | Sequence | (bp) | Reference |

| | | | |
|---|---|---|---|
| TIS7SA | CTCTTATCTCGGCATTTG | 342 | |
| | GGACAAGAGAAAGCAGCG | | |
| TIS7B | CGATGCCGAAGAACAAGA | 300 | |
| | CTGCCTGTCTTGTCTTCG | | |
| IFN-β | GAAAAGCAAGAGGAAAGATT | 165 | (Nickolaus& |
| | AAGTCTTCGAATGATGAGAA | | Zawatzky, 1994) |
| IFN-α | AATGACCTCCACCAGCAGCT | 201 | (Nickolaus& |
| | TCTCAGGTACACAGTGATCC | | Zawatzky, 1994) |
| IFN-α/βR | ACATGAGCCCCCCAGAAGTACG | 613 | (Kita et |
| | ATGACCGGAGGAGGAGGGAGAA | | al., 1994) |
| IRF-1 | CAGAGGAAAGAGAGAAGTCC | 201 | (Barber et |
| | CACACGGTGACAGTGCTGG | | al., 1995) |
| IRF-2 | CCTGAGTATGCGGTCCTGACTT | 528 | (Kita et |
| | CCGGGTCTCCCGGTCTGGCCGA | | al., 1994) |
| TNF-α | GAAAGCATGATCCGCGACGTGG | 678 | (Kita et |
| | GTAGACCTGCCCGGACTCCGCAA | | al, 1994) |
| β-Actin | ATAGCACAGCTTCCCTTTGAT | 452 | (Tal-Singer et |
| | AACATGCATTGTTACCAACT | | al., 1997) |
| Cyclophilin | ATTCGAGTTGTCCACAGTCAGCAATGG | 469 | (Bergsma et |
| | ATGGTCAACCCCCACCGTGTTCTTCGAC | | al., 1991) |

Primers specific for TIS7 were designed based on the published sequence (Varnum et al., 1989). Cycling reactions were performed with a Perkin Elmer Cetus Gene Amp PCR System thermocycler. After one cycle of 9 min denaturation at 94° C., cycles were as follows: (i) 1 min denaturation at 94° C., (ii) annealing at 60° C. for 1 min, (iii) extension for 2 min at 72° C. The final cycle was terminated with a 7 min extension at 72° C. Amplification was carried out for 25-35 cycles. RT reactions were included in each set of experiments as negative controls, and 10 ng of mouse DNA was used as a positive control. In every case, the size of PCR product bands corresponded to the predicted MW.

### Example 7

### Detection of PCR Products

Aliquots of 40% of the amplification products were fractionated on 2.5% NuSieve Agarose (FMC). Gels were stained with ethidium bromide (Sigma) and the amount of products were quantitated by fluorimetry. The relative amount of PCR product was determined in arbitrary numbers as the ratio between the PCR product band intensity to that of cellular housekeeping genes cyclophilin or β-actin (Devi-Rao et al., 1994, Tal-Singer et al., 1997). Statistical analysis was performed using Microsoft Excel (Redmond, Wash.).

### Example 8

### Immunohistochemical Procedures

Ganglia used for immunohistochemistry were fixed for 24 h with 4% paraformaldehyde in PBS then immersed in 70% ethanol/150 mM NaCl for 24 h and embedded in paraffin wax, and 6 mm serial sections were cut and processed as described elsewhere (Randazzo et al., 1995). Rabbit polyclonal antisera to HSV-1 (Dako Corporation, Carpinteria, Calif.) was used for detection of replicating virus as described (Adams et al., 1984, Kesari et al., 1995). Rabbit polyclonal anti-mouse interferon-a/b (Lee Biomolecular Research, San Diego, Calif.), and rabbit polyclonal anti-mouse TNF-a (Genzyme Diagnostics, Cambridge, Mass.) were used to probe for cytokines. Rabbit polyclonal anti-TIS7 was a generous gift from B. Varnum, Amgen, Calif. Antigen-expressing cells were detected by an indirect avidin-biotin immunoperoxidase method (Vectastain ABC kit, Vector Labs, Burlingham, Calif.), with 3,3′-diaminobenzadine (DAB) as the chromagen (Trojanowski et al., 1993).

We have recently shown that cellular Immediate Early factors, such as c-jun, c-myc, and Oct-1, are induced in neuronal cells at early times following explantation of latent HSV-1-infected murine ganglia (Tal-Singer et al., 1997, Valyi-Nagy et al., 1991). DDRT-PCR was used in the present study as an approach to identify previously unknown cellular genes which are induced or repressed by explantation of latently-infected TG. This method allows the visualization and subsequent isolation of cDNAs corresponding to mRNAs that are differentially expressed in various cell populations (Liang & Pardee, 1992).

### Example 9

### Explantation of TG Induces Differential Expression of Multiple mRNAs

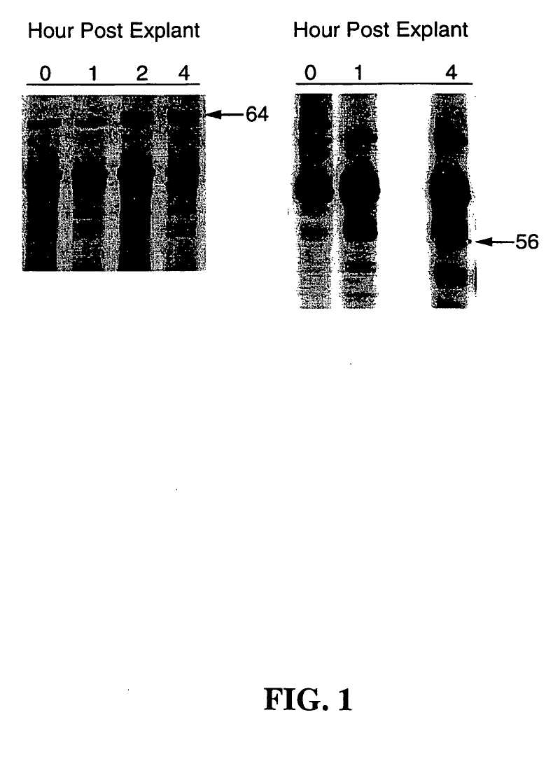

RNA was prepared from latently-infected TG at different time points (0, 1, 2, and 4 hours) following explantation into culture media. Complementary DNA was amplified using a set of arbitrary PCR primers. The PCR products were resolved by PAGE and visualized by autoradiography. Every pair of primers (216 primer combinations) identified a limited number of target sequences within the pool of cDNAs. Thus, a typical reaction generated 50-200 distinct radiolabeled PCR products between 50 and 600 bp in length. As expected from previous studies (Liang et al., 1995), the majority of PCR products were present at identical levels in samples derived from different time points (FIG. 1). However, over 100 differentially-displayed PCR products were detected and isolated. All differentially-displayed products were isolated for further characterization. Four overlapping products (#56, #64, #116, and #125 as shown in FIG. 2B) are described in this report. The intensity of the four PCR products was clearly increased in samples prepared 1 and 2 hours after explantation (#56, and #64 shown in FIG. 1).

Reamplified PCR products were subjected to sequencing followed by BLAST (Altschul et al., 1990) sequence analysis. The sequences of bands #56, #64, #116, and #125 were identical to sequences in the coding region of mouse TIS7 mRNA (Varnum et al., 1989) ( FIG. 2), and were conserved with rat PC4 mRNA (Tirone & Shooter, 1989). TIS7 and rat PC4 were previously shown to be highly related in protein sequence and are thought to be functional homologs (Tirone & Shooter, 1989, Varnum et al., 1989).

### Example 10

### Confirmation of Differential Display

We next determined whether RNA corresponding to the isolated bands was differentially expressed in either uninfected or latently-infected TG explants. Reamplified PCR products were used as probes in quantitative RNase protection assays (RPA). RNA corresponding to band #56 was induced by 2 hours following explantation of uninfected (FIG. 3A) and infected (results not shown) explants. Phosphorimager quantitation (FIG. 3B) indicated that the levels of RNA were induced nearly 7-fold by four hours. Similar results were obtained using probes generated from band #64 (results not shown). Thus, RNA transcripts corresponding to differentially-displayed bands #56, and #64 were clearly induced in TG by explantation.

To further confirm the DDRT results, cDNA prepared from a different set of latently infected TG explants was subjected to PCR using primers specific for TIS7 (Table 3. Each PCR reaction yielded single product bands whose expected sizes corresponded to TIS7 cDNA. Importantly, TIS7 detected with these primers was rapidly induced in both infected (FIG. 3C) and uninfected explants (not shown), confirming the RPA results (FIG. 3A). Furthermore, by 24 hours p.e., TIS7 expression returned to basal levels (FIG. 3C). We also confirmed TIS7 induction in neuronal cells by immunostaining using affinity purified polyclonal antisera directed against TIS7 (not shown). Thus, induction of TIS7 following explantation of ganglia was detected by three independent methods: DDRT-PCR, RPA, and immunostaining.

### Example 11

### IFN-β is Induced by Explantation of TG

Several reports have indicated that TIS7/PC4 are related in sequence to IFN-β (Skup et al., 1982, Tirone & Shooter, 1989). To determine whether interferons are also induced by explantation, we again used qualitative RT-PCR. cDNA derived from infected and uninfected TG explants was analyzed by PCR using primers specific for IFN-α or IFN-β (Table 3. Again, single specific PCR products were obtained from each reaction. IFN-β was induced 2-3.5-fold during the four hours following explantation, as compared to the amount of cellular housekeeping gene cyclophilin (FIG. 4A). Similarly, IFN-α levels were 1.2-1.4-fold higher (not shown). Similar results were obtained for uninfected samples (results not shown).

### Example 12

### IFN Expression is Induced in Neuronal Cells

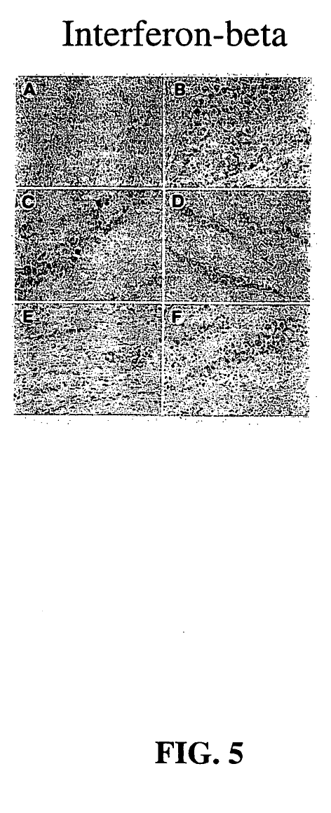

HSV latency occurs primarily in neuronal cells which innervate the cornea (Cook et al., 1974, Ramakrishnan et al., 1994, Ramakrishnan et al., 1996). These neurons represent approximately 2% of the neuronal population in the TG (Arvidson, 1977, Marfurt et al., 1989). To determine whether IFN expression co-localizes with reactivating virus in neuronal cells, TG sections from latently-infected and uninfected explants were analyzed by immunohistochemistry. IFN α/β protein expression was not detected at 0 h p.e., and was induced at 4, 8, and 24 h p.e. in both infected and uninfected explants (FIG. 5). Furthermore, as judged by this technique, all of the neuronal cells expressed IFN. Since virus reactivates in approximately 1% of neuronal cells (Valyi-Nagy et al., 1991), we conclude that viral gene expression occurs in cells expressing IFN.

### Example 13

### Induction of Interferon Regulatory Factor-1 (IRF-1)

The IFN regulatory factors (IRF) bind to interferon consensus sequences (ICSs) found in many promoters of IFN gene family members (Herschman, 1991, Tanaka & Taniguchi, 1992, Taniguchi et al., 1995). IRF-1 is an activator of IFN-b, whereas IRF-2 functions as a repressor (Watanabe et al., 1991). As shown in FIG. 4B, IRF-1 transcription was induced within the first hour p.c., and its profile of induction was strikingly similar to IFN-β (FIG. 4A). In contrast, no significant change was observed in the levels of the IFN-α and β receptor (IFNαβR or IRF-2 (FIGS. 4 C, 4D). Induction of IFN and IRF-1 also occured in the absence of serum in the explantation media (not shown), indicating that serum factors are not the cause for our observations. These results were reproducible in both infected and uninfected preparations (not shown), as also found for TIS7 (FIG. 3). Our results suggest that IFN induction after explantation of TG involves an IRF-1-dependent pathway.

### Example 14

### TNF-a is Induced by TG Explantation

Soluble tumor necrosis factor (TNF)-a enhances the reactivation frequency and replication of HSV-1 during explant reactivation (Walev et al., 1995). To determine whether endogenous TNF-α is induced during explantation, RT-PCR was performed using primers specific for TNF-α. TNF-α transcripts were induced rapidly following explant (FIG. 6). However, we were unable to detect TNF-α, a secreted factor, in TG sections by immunostaining.

### Example 15

### Oligonucleotides, Probes, and Electromobility Shift Assays

To generate probes for gel mobility shift assays and competition experiments, single-stranded oligo nucleotides were synthesized (Gibco BRL Life Technologies) annealed with complementary oligonucleotides and labeled as described previously (Scahffer et al., 1997, Frazier et al., 1996). The sequence of each probe and its mutant derivatives is descibed below:

| | |
|---|---|
| ISRE | 5′ GATCCTAGAAGGGAAACCGAACTGAGGATC |
| LAT | 5′ GATCGAGGGGAAAAGTGAAAGACACGGGCA |
| LATmAT | 5′ GATCGAGGGGAAAAGATAAAGACACGGGCA |
| LATmm | 5′ GATCGAGGGGAAAAGTGGCTGACACGGGCA |
| OriS | 5′ GATCATTATAAAAAAAGTGCGAACGCGAG |

DNA binding assays were carried out as described previously (Frazier et al., 1996). The reaction was in 20 ul total volume containing 2 ul (20 ug) BSA (NEB) 0.2 ul (2 ug) sheared salmon sperm DNA9 ul buffer D (20 mM herpes, pH7.9, 100 mM KCl, 20% glycerol, 0.2 mM EDTA, 1 mM DT7, protein (usually 1-2 ul) and water. In samples where antibody incunbation was added, the reaction was incubated with unlabeled probe (100 fold) at room T for 20 minutes before the probe (<1 ng, 30K cpm) being added. Reaction was done at RT for 20 minutes. For supershift, antiserum (Santa Cruz) was added and incubated for one hour at RT. The gels were run at 4C at 170 V for about 2-3 hours. Gels were transfered to 3M paper, dried and exposed to X-ray film.

### Example 16

### In Vitro Translated IRF-1 Binds to LAT ISRE

IRF-1 protein sample was generated by in vitro transcription and translation of a human IRF-1 DNA template (generous gift from Richard Pine, New York University) by using the rabbit reticulate lysate system (Promega) and incubated with LAT probe. As shown in FIG. 8, IRF-1 formed a complex LAT ISRE DNA sequence. This complex was supershifted with IRF-1 specific antisera but not with IRF-2 antisera, indicating the specificity of the interaction. We have been unable to show in gel mobility shift assays competition experiments with oriL site III oligo, and oriS site I oligo possess IRF binding sites (not shown). However, the LAT region ISRE was shown to be able to associate with IRF-2 in phorbol ester stimulated Jurkat cells extracts (Santa Cruz) in gel mobility shift assays (not shown). Moreover, the IRF-2/ISRE complex (Complex B) formed when the LAT probe was incubated with Jurkat cell extracts was competed by wild-type LAT and ISRE probes (Santa Cruz) but not by mutant ISRE mQpinr (Santa Cruz), mutated LAT probes LATmAT and LATmm that contain two and three nucleotide changes, respectively, or and oriS Site I oligo (FIG. 9). These experiments indicate that the LAT site is truly an IRF binding site capable of binding both IRF-1 and IRF-2.

### Example 17

### Induction of TIS7 and IRF-1 is Induced by Hyperthermia

Transient hyperthermia is known to cause reactivation of HSV-1 from latency (Sawtell et al., 1992). To determine whether IFN-related genes such as TIS7 and IRF-1 are induced by another reactivation stimulus, samples from individual hyperthermia-treated mice were prepared. As shown in FIG. 10A, cellular housekeeping genes were realtively similar in all the animals, whereas HSP70, TIS7, and IRF-1 transcription was induced within the first 1-2 hours following hyperthermia (FIGS. 10 B, C, D). Similar results were obtained in two experiments using samples obtained from different mice (not shown). Our results suggest that the pathway leading to induction of TIS7 and IRF-1 is common to different reactivation stimuli.

### REFERENCES

The full references listed below are cited thoughout this patent application.
Adams, R. L., Springall, D. R., Levene, M. M. & Bushell, T. E. (1984). The immunocytochemical detection of herpes simplex virus in cervical smears-a valuable technique for routine use. J. Pathol. 143(4), 241-247.
Altschul, S. F., Gisg, W., Miller, W., Meyers, E. W. & Lipman, D. j. (1990). BLAST: a local alignment search tool. J. Mol. Biol. 215, 403-410.
Arenalder, A. T., Lim, R. W., Varnum, B. C., Cole, R., Vellis, J. d. & Herschman, H. R. (1989). TIS gene expression in cultured rat astrocytes: induction by mitogens and stellation agents. J. of Neuroscience Research 23, 247-256.
Arvidson, B. (1977). Retrograde axonal transport of horseradish peroxidase from cornea to trigeminal ganglion. Acta Neuropathologica 38, 49-52.
Barber, S. A., Fultz, M. J., Salkowski, C. A. & Vogel, S. N. (1995). Differential expression of interferon regulatory factor 1 (IRF-1), IRF-2, and interferon consensus sequence binding protein genes in lipopolysaccharide (LPS)-responsive and LPS-hyporesponsive macrophages. Infect. Immunol. 63, 601-608.
Bergsma, D. J., Eder, C., Gross, M., Kersten, H., Sylvester, D., Appelbaum, E., Cusiamano, D., Levi, G. P., McLaughlin, M. M., Kasyan, K., Prichett, W. P., Bossard, M. J., Brandt, M. & Levy, M. A. (1991). The cyclophilin multigene family of peptidyl-prolyl isomerases: characterization of three separate human isoforms. J. Biol. Chem. 266, 23204-23214.
Brown, S. M., Ritchie, D. A. & Subak-Sharpe, J. (1973). Genetic studies with herpes simplex virus type 1. The isolation of temperature-sensitive mutants, their arrangement into complementation groups and recombination analysis leading to lineage groups. J. Gen. Virol. 18, 329-346.
Chatterjee, S., Hunter, E. & Whitley, R. (1985). Effect of cloned human interferons on protein synthesis and morphogenesis of herpes simplex virus. J. Virology 56, 419-425.
Cook, M. L., Bastone, V. B. & Stevens, J. B. (1974). Evidence that neurons harbor latent herpes simplex virus. Infect. Immun. 9, 946-951.
Dabrowski, C. E., Carmillo, P. J. & Schaffer, P. A. (1994). Cellular protein interactions with herpes simplex virus type 1 oriS. Molecular & Cellular Biology 14, 2545-55.
Dabrowski, C. E. & Schaffer, P. A. (1991). Herpes simplex virus type 1 origin-specific binding protein:oriS-binding properties and effects of cellular proteins. Journal of Virology 65, 3140-50.
Devi-Rao, G. B., Bloom, D. C., Stevens, J. G. & Wagner, E. K. (1994). Herpes simplex virus type I DNA replication and gene expression during explant-induced reactivation of latently infected murine sensory ganglia. J. Virol 68, 1271-1282.
Drew, P., Franzoso, G., Carlson, L., Biddison, W., Siebenlist, U. & Ozato, K. (1995). Interferon regulatory factor-2 physically interacts with NF-kappa B in vitro and inhibits NF-kappa B induction of major histocompatibility class I and beta 2-microglobulin geneexpression in transfected human neuroblastoma cells. J Neuroimmunol 63.
Fawl, R. L., Gesser, R. M., Valyi-Nagi, T. & Fraser, N. W. (1996). Reactivation of herpes simplex virus from latently infected mice after administration of cadmium is mouse-starin-dependent. J. Gen. Virol. 77, 2781-2786.
Fawl, R. L. & Roizman, B. (1993). Induction of reactivation of herpes simplex virus in murine sensory ganglia in vivo by cadmium. J. Virol 67, 7025-7031.
Fraser, N. W., Spivack, J. G., Wroblewska, Z., Block, T., Deshmane, S. L., Valyi-Nagy, T., Natarajan, R. & Gesser, R. (1991). A review of the molecular mechanism of HSV-1 latency. Curr. Eye Res. 10 (Suppl), 1-14.
Fraser, N. W. & Valyi-Nagy, T. (1993). Viral, neuronal and immune factors which may influence herpes simplex virus (HSV) latency and reactivation. Microbial Pathogen 15, 83-91.
Frazier, D. P., D. Cox, E. M. Godshalk, & P. Schaffer. (1996). Identification of cis-acting sequences in the promoter of the Herpes simplex virus type 1 latency-associated transcripts required for activation by nerve growth factor and sodium butyrate in PC12 cells. J. Virol. 70,7433-7444.
Gobl, A. E., Funa, K. & Alm, G. V. (1988a). Different induction patterns of mRNA for IFN-alpha and -beta in human mononuclear leukocytes after in vitro stimulation with herpes simplex virus-infected fibroblasts and Sendai virus. Journal of Immunology 140, 3605-9.
Gobl, A. E., K. Funa & Alm, G. V. (1988b). Different induction patterns of mRNA for IFN-alpha and -beta in human mononuclear leukocytes after in-vitro stimulation with herpes simplex virus-infected fibroblasts and Sendai virus. J. Immunology 140, 3605-3609.
Green, J. A., Yeh, T. J. & Overall, J. C., Jr. (1981). Sequential production of IFN-alpha and immune-specific IFN-gamma by human mononuclear leukocytes exposed to herpes simplex virus. Journal of Immunology 127, 1192-6.
Green, J. A., Yeh, T. J. & Overall, J. C. (1991). Sequential production of IFN-alpha and immune-specific IFN-gamma by human mononuclear leukocytes exposed to herpes simplex virus. J. Immunology 127, 1192-1198.
Gstaiger, M., Knoepfel, L., Georgiev, O., Schaffner, W. & Hovens, C. M. (1995). A B-cell coactivator of octamer-binding transcription factors. Nature 373, 360-2.
Guardavaccaro, D., Ciotti, M. T., Schafer, B. W., Montagnoli, A. & Tirone, F. (1995). Inhibition of differentiation in myoblasts deprived of the interferon-related protein PC4. Cell Growth and Differentiation 6, 159-169.
Habu, S., Akamatsu, K., Tamaoki, N. & Okumura, K. (1984). In vivo significance of NK cells on resistance against virus (HSV-1) infections in mice. J. Immunology 133, 2743-2747.
Harbour, D. A., Hill T. J., & Blythe W. A. (1981). Acute and recurrent herpes simplex in several strains of mice. J. Gen. Virol. 55, 31-40.
Harwick, J., Romanowski, E., Araullo-Cruz, T. & Gordon, Y. J. (1987). Timolol promotes reactivation of latent HSV-1 in the mouse iontophoresis model. Investigative Opthalmology and Visual Science 28.
Herschman, H. R. (1991). Primary response genes induced by growth factors and tumor promoters. In Annual Review of Biochemistry, pp. 281-319. Edited by C. C. Richardson, J. N. Abelson, A. Meister & C. T. Walsh. Palo Alto, Calif.: Annual Reviews, Inc.
Herschman, H. R., Kujubu, D. A., Fletcher, B. S., Ma, Q., Varnum, B. C., Gilbert, R. S. & Reddy, S. T. (1994). The tis genes, primary response genes induced by growth factors and tumor promoters in 3T3 cells. Progress in Nucleic Acid Research & Molecular Biology 47, 113-48.
Hill, T. J. (1985). Herpes simplex virus latency. In The Herpes Viruses, pp. 175-240. Edited by B. Roizman. New York: Plenum Publishing Corp.
Hobart, M., Ramassar, V., Goes, N., Urmson, J. & Halloran, P. (1997). IFN regulatory factor-1 plays a central role in the regulation of the expression of class I and II MHC genes in vivo. J. Immunol. 158, 4260-4269.
Honda, Y., Kawai, A. & Matsumoto, S. (1985). Persistent infection of rabies virus (HEP-Flury strain) in human neuroblastoma cells capable of producing interferon. J. Gen. Virol. 66, 957-967.
Inokuchi, K., Murayama, A. & Ozawa, F. (1996). mRNA differential display reveals Krox-20 as a neural plasticity-regulated gene in the rat hippocampus. Biochemical & Biophysical Research Communications 221, 430-6.
Jahner, D. & Hunter, T. (1991). The stimulation of quiescent rat fibroblasts by v-src and v-fps oncogenic protein-tyrosine kinases lead to the induction of a subset of immediate early genes. Oncogene 6, 1259-1268.
Kawakami, T., Matsumoto, M., Sato, M., Harada, H., Taniguchi, T. & Kitagawa, M. (1995). Possible involvement of the transcription factor ISGF3 gamma invirus-induced expression of the IFN-beta gene. FEBS Letters 358, 225-9.
Kesari, S., Randazzo, B. P., Valyi-Nagy, T., Huang, Q. S., Brown, S. M., MacLean, A. R., Lee, V. M.-Y., Trojanowski, J. Q. & Fraser., N. W. (1995). Therapy of experimental human brain tumors using a neuroattenuated herpes simplex virus mutant. Lab Invest 73, 636-648.
Kiryu, S., Yao, G. L., Morita, N., Kato, H. & Kiyama, H. (1995). Nerve injury enhances rat neuronal glutamate transporter expression: identification by differential display PCR. J. Neuroscience 15, 7872-8.
Kita, M., Tanaka, K., Shinmura, K., Tanaka, Y., Liu, Y. & Imanishi, J. (1994). Expression des genes des cytokines et des genes associes a l'interferon chez l'embryon de la souris. C. R. Soc. Biol. 188, 593-600.
Kondo, T. (1996). Identification and analysis of IRF-1 binding protein. Hokkaido Igaku Zasshi 71, 509-516.
Kujubu, D. A., Lim, R. W., Varnum, B. C. & Herschman, H. R. (1987). Induction of transiently expressed genes in PC-12 pheochromocytoma cells. Oncogene 1, 257-262.
LaMarco, K. & McKnight, S. (1989). Purification of a set of cellular polypeptides that bind to the purine-rich cis-regulatory element of herpes simplex virus immediate early genes. Genes and Development 3, 1372-1383.
Leblanc, J. F. & Hiscott, J. (1992). Differential response of human interferon-beta promoter. Virology 186, 760-3.
Liang, P., Bauer, D., Averboukh, L., Warthoe, P., Rohrwild, M., Muller, H., Strauss, M. & Pardee, A. B. (1995). Analysis of altered gene expression by differential display. In Methods in Enzymology: Oncogene Techniques, pp. 304-321. Edited by P. K. Vogt & I. M. Verma. New York: Academic Press.
Liang, P. & Pardee, A. B. (1992). Differential Display of eukaryotic messenger RNA by means of the polymerase chain reaction. Science 257, 976-97 1.
Lim, R. W., Varnum, B. C. & Herschman, H. R. (1987). Cloning of tetradecanoyl phorbol ester-induced ‘primary response’ sequences and their expression in density-arrested Swiss 3T3 cells and a TPA non-proliferative variant. Oncogene 1, 263-70.
Luo, Y. & Roeder, R. G. (1995). Cloning, functional characterization, and mechanism of action of the B-cell-specific transcriptional coactivator OCA-B. Molecular & Cellular Biology 15, 4115-24.
MacDonald, N. J., Kuhl, D., Maguire, D., Naf, D., Gallant, P., Goswamy, A., Hug, H., Bueler, H., Chaturvedi, M. & Fuente, J. d. l. (1990). Different pathways mediate virus inducibility of the human IFN-alpha 1 and IFN-beta genes. Cell 60, 767-79.
Maniatis, T., Fritsch, E. F. & Sambrook, J. (1982). Molecular cloning: a laboratory manual. Cold Spring Harbor, N.Y.: Cold Spring Harbor Laboratory.
Marfurt, C. F., Kingsley, R. E. & Echtenkamp, S. E. (1989). Sensory and sympathetic innervation of the mammalian cornea. Investigative Opthalmol. Visual. Sci. 30, 461-472.
Nickolaus, P. & Zawatzky, R. (1994). Inhibition by interleukin-4 of constitutive beta interferon synthesis in mouse macrophages. J. Virol. 68, 6763-6766.
Nonkwelo, C., I. K. Ruf, and J. Sample. 1997. Interferon-independent and induced regulation of Epstein-Barr virus EBNA-1 gene transcription in Burkitt lymphoma J. Virol. 71, 6886-6897.
O'Hare, P. & Goding, C. R. (1988). Herpes simplex virus regulatory elements and the immunoglobulin octamer bind a common factor and are both targets for virion transactivation. Cell 52, 435-445.
Openshaw, H., Asher, S., Wohlenberg, C., Sekizawa, T. & Notkins, A. L. (1979). Acute and latent infection in sensory ganglia with herpes simplex virus: Immune control and virus reactivation. J. Gen. Virol 44, 205-215.
Pine, R., Canova, A. & Schindler, C. (1994). Tyrosine phosphorylated p91 binds to a single element in the ISGF2/IRF-1 promoter tomediate induction by IFN alpha and IFN gamma, and is likely to autoregulate the p91 gene. EMBO J 13.
Qu, D., Ludwig, D. S., Gammeltoft, S., Piper, M., Pelleymounter, M. A., Cullen, M. J., Mathes, W. F., Przypek, R., Kanarek, R. & Maratos-Flier, E. (1996). A role for melanin-concentrating hormone in the central regulation of feeding behaviour. Nature 380, 243-7.
Ramakrishnan, R., Levine, M. & Fink, D. J. (1994). PCR-based analysis of herpes simplex virustype 1 latency in the rat trigeminal ganglion established with a ribonucleotide reductase-deficient mutant. Journal of Virology 68, 7083-91.
Ramakrishnan, R., Poliani, P. L., Levine, M., Glorioso, J. C. & Fink, D. J. (1996). Detection of herpes simplex virus type 1 latency-associated-associated transcript expression in trigeminal ganglia by in situ reverse transcriptase PCR. Journal of Virology 70, 6519-23.
Randazzo, B. P., Kesari, S., Gesser, R. M., Alsop, D., Ford, J. C., Brown, S. M., MacLean, A. R. & N. W. Fraser (1995). Treatment of experimental intracranial murine melanoma with a neuro-attenuated Herpes Simpex virus-1 mutant. Virol 211, 94-101.
Roizman, B. (1991). Herpesviridae: a brief introduction. In Fundamental virology, pp. 841-847. Edited by B. N. Fields & D. M. Knipe. New York: Raven Press, Ltd.
Roizman, B. & Sears, A. E. (1987). An inquiry into the mechanisms of herpes simplex virus latency. Ann. Rev. Microbiol. 41, 543-571.
Sawtell, N. M. & Thompson, R. L. (1992). Rapid in vivo reactivation of herpes simplex virus in latently infected murine ganglionic neurons after transient hyperthermia. J. Virol. 66, 2150-2156.
Schaffer, B. C., E. Paulson, J. L. Strominger, and S. H. Speck. 1997. Constitutive activation of Epstein-Barr virus (EBV) nuclear antigen 1 gene transcription by IRF1 and IRF2 during restricted EBV latency. Mol. Cel. Biol. 17,873-886.
Sheng, M. & Greenberg, M. E. (1990). The regulation and function of c-fos and other immediate early genes in the nervous system. Neuron 4, 477-485.
Skup, D., Windass, J. D., Sor, F., George, H., Williams, B. R., Fukuhara, H., Maeyer-Guignard, J. D. & Maeyer, E. D. (1982). Molecular cloning of partial cDNA copies of two distinct mouse IFN-beta mRNAs. Nucleic Acids Res. 10, 3069-3084.
Sompayrac, L., Jane, S. & Danna, K. J. (1996). Reduced levels of alpha1(XI) procollagen mRNA in SV40-transformed cells. Virology 218, 412-6.
Stanwick, T. L., Campbell, D. E. & Nahmias, A. J. (1982b). Cytotoxic properties of human monocyte-macrophages for human fibroblasts infected in herpes simplex virus: interferon production and augmentation. Cellular Immunology 70, 132-47.
Stanwick, T. L., Campbell, D. E. & Nahmias, A. J. (1982a). Cytotoxic properties of human monocyte-macrophages for human fibroblasts infected in herpes simplex virus: interferon production and augmentation. J. Cell. Immunol. 70, 132-139.
Stevens, J. G. (1989). Human herpesviruses: A consideration of the latent state. Microbial Rev. 53, 318-332.
Strubin, M., Newell, J. W. & Matthias, P. (1995). OBF-1, a novel B cell-specific coactivator that stimulates immunoglobulin promoter activity through association with octamer-binding proteins. Cell 80, 497-506.
R. Tal-Singer, T. M. Lasner, W. Podrzucki, A. Skokotas, J. J. Leary, S. L. Berger, and N. W. Fraser. (1997). Gene expression during reactivation of herpes simplex type 1 from the peripheral nervous system is different from that during lytic infection of tissue culture. Journal of Virology, 71:5268-5276.
R. Tal-Singer, W. Podrzucki, T. M. Lasner, A. Skokotas, J. J. Leary, S. L. Berger, and N. W. Fraser. (1998). Diffrential expression of Interferon-β and its associated genes in neuronal cells following explant of mouse trigeminal ganglia: potential role in reactivation of HSV-1 from latency. Journal of Virology, 72:1252-1261.
Tamura, T., Ueda, S., Yoshida, M., Matsuzaki, M., Mohri, H. & Okubo, T. (1996). Interferon-gamma induces Ice gene expression and enhances cellular susceptibility to apoptosis in the U937 leukemia cell line. Biochem Biophys Res Commun 229, 21-26.
Tanaka, N., Ishihara, M., Lamphier, M., Nozawa, H., Matsuyama, T., Mak, T., Aizawa, S., Tokino, T., Oren, M. & Taniguchi, T. (1996). Cooperation of the tumour suppressors IRF-1 and p53 in response to DNA damage. Nature 382, 816-818.
Tanaka, N. & Taniguchi, T. (1992). Cytokine gene regulation: regulatory cis-elements and DNA binding factors involved in the interferon system. In Advances in Immunology, pp. 263-281. San Diego, Calif.: Academic Press.
Taniguchi, T., Harada, H. & Lamphier, M. (1995). Regulation of the interferon system and cell growth by the IRF transcription factors. Journal of Cancer Research & Clinical Oncology 121, 516-20.
Tirone, F. & Shooter, E. (1989). Early gene regulation by nerve growth factor in PC12 cells:induction of an interferon-related gene. PNAS 86, 2088-2092.
Trojanowski, J., Mantione, R., Lee, J., Seid, D., You, T., Inge, L. & Lee, V. (1993). Neurons Derived from a Human Teratocarcinoma Cell Line Establishes Molecular and Structural Polarity Following Transplantation into the Rodent Brain. Experimental Neurology 122, 283-294.
Valyi-Nagy, T., Deshmane, S. L., Dillner, A. J. & Fraser, N. W. (1991). Induction of cellular transcription factors in trigeminal ganglia of mice by corneal scarification, herpes simplex virus 1 type infection and explantation of trigeminal ganglia. J. Virol 65, 4142-4152.
Varnum, B. C., Lim, R. W. & Herschman, H. R. (1989). Characterization of TIS7, a gene induced in Swiss 3T3 cells by the tumor promoter tetradecanoyl phorbol acetate. Oncogene 4, 1263-1265.
Varnum, B. C., Reddy, S. T., Koski, R. A. & Herschman, H. R. (1994). Synthesis, degradation, and subcellular localization of proteins encoded by the primary response genes TIS7/PC4 and TIS21/PC3. J. of Cellular Physiology 158, 205-213.
Walev, I., Podlech, J. & Falke, D. (1995). Enhancement by TNF-a of reactivation and replication of latent herpes simplex virus from trigeminal ganglia. Arch. Virol. 140, 987-992.
Walz, M. A., Yamamoto, H. & Notkins, A. L. (1976). Immunological response restricts number of cells in sensory ganglia infected with herpes simplex virus. Nature 264, 554-559.
Wang, I., Blanco, J., Tsai, S., Tsai, M. & Ozato, K. (1996). Interferon regulatory factors and TFIIB cooperatively regulate interferon-responsive promoter activity in vivo and in vitro. Mol Cell Biol 16, 6313-6324.
Warthoe, P. (1995). Detection and Identification of Expressed Genes by Differential Display. In PCR Primer: A Laboratory manual, pp. 421-438. Edited by C. W. Dieffenbach & G. S. Dveksler: Cold Spring Harbour Library Press.
Watanabe, N., Sakakibara, J., Hovanessian, A. G., Taniguchi, T. & Fujita, T. (1991). Activation of IFN-beta element by IRF-1 requires a posttranslational event in addition to IRF-1 synthesis. Nuc. Ac. Res. 19, 4421-8.
Willey, D. E., Trousdale, M. D. & Nesburn, A. B. (1984). Reactivation of murine latent HSV infection by epinephrine iontophoresis. Invest. Ophthal. Vis. Sci. 25, 945-950.
Yan, J., Khanna, K. K. & Lavin, M. F. (1996). Induction of inositol 1,4,5 trisphosphate receptor genes by ionizing radiation. International Journal of Radiation Biology 69, 539-40.

1. A method of treating viral infection or reactivation comprising the steps of:
contacting an individual with an antagonist of the interaction between a polynucleotide sequence comprising SEQ ID NO:1 and IRF-1, and
antagonizing said interaction.

2. The method of claim 1 wherein said viral infection or reactivation is from HSV-1, HSV-2, VZV, HCMV or EBV.

3. A method of treating viral infection or reactivation comprising the steps of:
contacting an individual with a first compound capable of lowering the level of a second compound, said second compound selected from the group consisting of IRF-1, TIS7, IFN-α and IFN-β, and
lowering said level.

4. The method of claim 3 wherein said viral infection or reactivation is from HSV-1, HSV-2, VZV, HCMV or EBV.

5. An isolated polynucleotide comprising a polynucleotide sequence selected from the group consisting of the polynucleotide sequences set forth in Table 3 and SEQ ID NO:1.

6. A composition comprising a Herpesvirus polypeptide involved in viral infection and/or reactivation.

7 The composition of claim 6 wherein said polypeptide binds specifically to a polynucleotide.

8. The composition of claim 7 wherein said polynucleotide comprises a polynucleotide sequence derived from the group consisting of SEQ ID NO:1, an IRF-1 binding site consensus sequence, a sequence comprising oriL, and a sequence comprising oriS.

9. A composition comprising IRF-1.

10. A method for screening for compounds capable of inhibiting specific binding of IRF-1 to a polynucleotide comprising:
providing a composition comprising IRF-1 specifically bound to a polynucleotide,
contacting said composition with a compound potentially capable of altering the binding of IRF-1 and said polynucleotide, and
detecting whether said binding is altered.

11. A compostion comprising an IRF-1: IRF-BP complex.

12. A method for for screening for compounds capable of inhibiting specific binding of IRF-1 to IRF-BP comprising:
providing a composition comprising IRF-1 bound to IRF-BP,
contacting said composition with a compound potentially capable of altering binding of IRF-1 and said IRF-BP, and
detecting whether said binding is altered.

13. The method for claim 12 whereby said altering is agonizing or antagonizing binding of IRF-1 and IRF-BP.

14. A compound capable of agonizing or antagonizing any compound in IRF-1 and/or interferon genetic regulatory pathway.

15. A method for treating a viral infection or reactivation comprising the steps of:
contacting an individual suspected of being infected with virus with a compound capable of agonizing or antagonizing any compound in IRF-1 and/or interferon genetic regulatory pathway.

16. A composition comprising an HSV IRF-1 binding site consensus sequence.

17. A method for treating viral infection comprising:
contacting an individual suspected of being infected with virus with a composition comprising a compound potentially capable of altering the specific binding of IRF-1 and a polynucleotide.

18. A method for treating viral infection comprising:
contacting an individual suspected of being infected with virus with a composition comprising a compound capable of altering the specific binding of IRF-1 and IRF-BP.

19. The method for claim 17 whereby said altering is agonizing or antagonizing binding of IRF-1 and IRF-BP.


SEQUENCE LISTING (145 sequences)

SEQ ID NO: 1 - DNA, 93 bp, Herpes simplex virus
aaaaaagtgagaacgcgaagcgttcgcactttgtcctaataatatatatattattaggacaaagtgcgaacgcttcgcgttctcacttttttt

SEQ ID NO: 2 - DNA, 16 bp, Herpes simplex virus
gggaaaagtgaaagac

SEQ ID NO: 3 - DNA, 16 bp, Herpes simplex virus
gacgtaagtgacgtcg

SEQ ID NO: 4 - DNA, 16 bp, Herpes simplex virus
aaaaaaagtgagaacg

SEQ ID NO: 5 - DNA, 16 bp, Herpes simplex virus
ccgccaagtgatcctg

SEQ ID NO: 6 - DNA, 22 bp, Herpes simplex virus
aaaagaagtgagaacgtcactt

SEQ ID NO: 7 - DNA, 16 bp, Herpes simplex virus
caccatcacttccacc

SEQ ID NO: 8 - DNA, 16 bp, Herpes simplex virus
cgttctcacttttttt

SEQ ID NO: 9 - DNA, 16 bp, Herpes simplex virus
ggtattcacttaccgc

SEQ ID NO: 10 - DNA, 16 bp, Herpes simplex virus
gtctttcacttttccc

SEQ ID NO: 11 - DNA, 16 bp, Herpes simplex virus
cgttctcacttctttt

SEQ ID NO: 12 - DNA, 16 bp, Cytomegalovirus
gccgaaagtgaaagta

SEQ ID NO: 13 - DNA, 16 bp, Cytomegalovirus
gaaagaagtgaaaccc

SEQ ID NO: 14 - DNA, 16 bp, Herpes simplex virus
cgcgcaagtgaagccg

SEQ ID NO: 15 - DNA, 16 bp, Herpes simplex virus
gttaaaagtgattttt

SEQ ID NO: 16 - DNA, 16 bp, Herpes simplex virus
caataaagtgagtcta

SEQ ID NO: 17 - DNA, 17 bp, Herpes simplex virus
dcaaataagtgataatg

SEQ ID NO: 18 - DNA, 16 bp, Herpes simplex virus
aaaagaagtgatacaa

SEQ ID NO: 19 - DNA, 16 bp, Herpes simplex virus
ggctaaagtgacagga

SEQ ID NO: 20 - DNA, 16 bp, Herpes simplex virus
cttcgaagtgaatatt

SEQ ID NO: 21 - DNA, 16 bp, Herpes simplex virus
gcagaaagtgatgtgg

SEQ ID NO: 22 - DNA, 16 bp, Herpes simplex virus
atcctaagtgaggtga

SEQ ID NO: 23 - DNA, 16 bp, Herpes simplex virus
ctacaaagtgaagaat

SEQ ID NO: 24 - DNA, 16 bp, Herpes simplex virus
agcctaagtgacggtg

SEQ ID NO: 25 - DNA, 16 bp, Herpes simplex virus
gtgtgtcacttgttgc

SEQ ID NO: 26 - DNA, 16 bp, Herpes simplex virus
caacttcacttcaaac

SEQ ID NO: 27 - DNA, 16 bp, Herpes simplex virus
aagattcacttaaagc

SEQ ID NO: 28 - DNA, 16 bp, Herpes simplex virus
tataatcactttccgc

SEQ ID NO: 29 - DNA, 16 bp, Herpes simplex virus
acgtatcactttcacg

SEQ ID NO: 30 - DNA, 16 bp, Herpes simplex virus
ggccatcacttcgggg

SEQ ID NO: 31 - DNA, 16 bp, Herpes simplex virus
tcgcatcacttaagaa

SEQ ID NO: 32 - DNA, 16 bp, Herpes simplex virus
atcgatcacttttttc

SEQ ID NO: 33 - DNA, 16 bp, Herpes simplex virus
cgacgtcactttgagc

SEQ ID NO: 34 - DNA, 16 bp, Herpes simplex virus
aaagttcacttattct

SEQ ID NO: 35 - DNA, 16 bp, Herpes simplex virus
gtttttcacttactga

SEQ ID NO: 36 - DNA, 16 bp, Herpes simplex virus
agaactcacttaagag

SEQ ID NO: 37 - DNA, 16 bp, Herpes simplex virus
aaaaatcacttttgga

SEQ ID NO: 38 - DNA, 16 bp, Herpes simplex virus
gtcgttcactttgccg

SEQ ID NO: 39 - DNA, 16 bp, Herpes simplex virus
gttaatcactttaagt

SEQ ID NO: 40 - DNA, 16 bp, Herpes simplex virus
ggatctcacttaccgc

SEQ ID NO: 41 - DNA, 16 bp, Herpes simplex virus
gccgttcacttttccg

SEQ ID NO: 42 - DNA, 16 bp, Herpes simplex virus
tccaaaagtgacttcg

SEQ ID NO: 43 - DNA, 16 bp, Herpes simplex virus
aagacaagtgaaccct

SEQ ID NO: 44 - DNA, 16 bp, Herpes simplex virus
gctgaaagtgaagtgg

SEQ ID NO: 45 - DNA, 16 bp, Herpes simplex virus
aaggaaagtgagaccg

SEQ ID NO: 46 - DNA, 16 bp, Herpes simplex virus
catacaagtgacaacc

SEQ ID NO: 47 - DNA, 16 bp, Herpes simplex virus
atgccaagtgatcggt

SEQ ID NO: 48 - DNA, 16 bp, Herpes simplex virus
ggttgaagtgaatgcc

SEQ ID NO: 49 - DNA, 16 bp, Herpes simplex virus
attttaagtgatccaa

SEQ ID NO: 50 - DNA, 16 bp, Herpes simplex virus
acgttaagtgaactta

SEQ ID NO: 51 - DNA, 16 bp, Herpes simplex virus
tatcaaagtgatttta

SEQ ID NO: 52 - DNA, 16 bp, Herpes simplex virus
ctgttaagtgagccaa

SEQ ID NO: 53 - DNA, 16 bp, Herpes simplex virus
tttataagtgaaacaa

SEQ ID NO: 54 - DNA, 16 bp, Herpes simplex virus
ttgcaaagtgaggtta

SEQ ID NO: 55 - DNA, 16 bp, Herpes simplex virus
acccaaagtgaacatc

SEQ ID NO: 56 - DNA, 16 bp, Herpes simplex virus
cagctaagtgacattt

SEQ ID NO: 57 - DNA, 16 bp, Herpes simplex virus
gttttaagtgactata

SEQ ID NO: 58 - DNA, 16 bp, Herpes simplex virus
gtgggaagtgaaacta

SEQ ID NO: 59 - DNA, 16 bp, Herpes simplex virus
agttttcactttcccc

SEQ ID NO: 60 - DNA, 16 bp, Herpes simplex virus
taatgtcactttgcat

SEQ ID NO: 61 - DNA, 16 bp, Herpes simplex virus
agttgtcacttttagc

SEQ ID NO: 62 - DNA, 16 bp, Herpes simplex virus
aactttcacttctttt

SEQ ID NO: 63 - DNA, 16 bp, Herpes simplex virus
ctacatcactttcgac

SEQ ID NO: 64 - DNA, 16 bp, Herpes simplex virus
gcccttcactttgtgg

SEQ ID NO: 65 - DNA, 16 bp, Herpes simplex virus
gaagttcacttagccc

SEQ ID NO: 66 - DNA, 16 bp, Herpes simplex virus
ttcattcactttggtc

SEQ ID NO: 67 - DNA, 16 bp, Herpes simplex virus
agacatcacttacgtg

SEQ ID NO: 68 - DNA, 16 bp, Herpes simplex virus
tataatcacttttctt

SEQ ID NO: 69 - DNA, 16 bp, Herpes simplex virus
ttgtatcacttacgat

SEQ ID NO: 70 - DNA, 16 bp, Herpes simplex virus
tcggatcactttataa

SEQ ID NO: 71 - DNA, 16 bp, Herpes simplex virus
aagcaaagtgaagggc

SEQ ID NO: 72 - DNA, 16 bp, Herpes simplex virus
gaccgaagtgaaggcc

SEQ ID NO: 73 - DNA, 16 bp, Herpes simplex virus
gaccgaagtgaaggcc

SEQ ID NO: 74 - DNA, 16 bp, Herpes simplex virus
gaccgaagtgaaggcc

SEQ ID NO: 75 - DNA, 16 bp, Herpes simplex virus
gaccgaagtgaaggcc

SEQ ID NO: 76 - DNA, 16 bp, Herpes simplex virus
gaccgaagtgaaggcc

SEQ ID NO: 77 - DNA, 16 bp, Herpes simplex virus
gaccgaagtgaaggcc

SEQ ID NO: 78 - DNA, 16 bp, Herpes simplex virus
gaccgaagtgaaggcc

SEQ ID NO: 79 - DNA, 16 bp, Herpes simplex virus
gaccgaagtgaaggcc

SEQ ID NO: 80 - DNA, 16 bp, Herpes simplex virus
gaccgaagtgaaggcc

SEQ ID NO: 81 - DNA, 16 bp, Herpes simplex virus
gaccgaagtgaaggcc

SEQ ID NO: 82 - DNA, 16 bp, Herpes simplex virus
tgtaaaagtgaagctg

SEQ ID NO: 83 - DNA, 16 bp, Herpes simplex virus
ctggaaagtgactcgg

SEQ ID NO: 84 - DNA, 16 bp, Herpes simplex virus
caagaaagtgaagcag

SEQ ID NO: 85 - DNA, 16 bp, Herpes simplex virus
gaagaaagtgaggaca

SEQ ID NO: 86 - DNA, 16 bp, Herpes simplex virus
agcacaagtgattagg

SEQ ID NO: 87 - DNA, 16 bp, Herpes simplex virus
ccctctcacttctact

SEQ ID NO: 88 - DNA, 16 bp, Herpes simplex virus
ggttgtcacttgtgag

SEQ ID NO: 89 - DNA, 16 bp, Herpes simplex virus
acagttcacttcctct

SEQ ID NO: 90 - DNA, 16 bp, Herpes simplex virus
acccctcactttgtac

SEQ ID NO: 91 - DNA, 16 bp, Herpes simplex virus
ataaatcacttcccta

SEQ ID NO: 92 - DNA, 16 bp, Herpes simplex virus
cggggtcacttcccct

SEQ ID NO: 93 - DNA, 16 bp, Herpes simplex virus
tggtgtcacttccgca

SEQ ID NO: 94 - DNA, 16 bp, Herpes simplex virus
atgcatcactttgagc

SEQ ID NO: 95 - DNA, 16 bp, Herpes simplex virus
gaccatcacttaagtt

SEQ ID NO: 96 - DNA, 16 bp, Herpes simplex virus
gataatcacttttatc

SEQ ID NO: 97 - DNA, 16 bp, Mus musculus
taggatcactttcata

SEQ ID NO: 98 - DNA, 10 bp, Mus musculus
gatcatagcc

SEQ ID NO: 99 - DNA, 10 bp, Mus musculus
ctgcttgatg

SEQ ID NO: 100 - DNA, 10 bp, Mus musculus
gatccagtac

SEQ ID NO: 101 - DNA, 10 bp, Mus musculus
gatcgcattg

SEQ ID NO: 102 - DNA, 10 bp, Mus musculus
aaactccgtc

SEQ ID NO: 103 - DNA, 10 bp, Mus musculus
tggtaaaggg

SEQ ID NO: 104 - DNA, 10 bp, Mus musculus
gatcatggtc

SEQ ID NO: 105 - DNA, 10 bp, Mus musculus
gttttcgcag

SEQ ID NO: 106 - DNA, 10 bp, Unknown
  This is missing from Primer #5
tacctaagcg

SEQ ID NO: 107 - DNA, 10 bp, Homo sapien
gatctgacac

SEQ ID NO: 108 - DNA, 10 bp, Mus musculus
gatctaaccg

SEQ ID NO: 109 - DNA, 10 bp, Unknown
  This is missing from Primer #5
tggattggtc

SEQ ID NO: 110 - DNA, 10 bp, Unknown
  This is missing from Primer #5
ggaaccaatc

SEQ ID NO: 111 - DNA, 10 bp, Mus musculus
gatcaatcgc

SEQ ID NO: 112 - DNA, 10 bp, Homo sapien
gatcaagtcc

SEQ ID NO: 113 - DNA, 10 bp, Homo sapien
gatctcagac

SEQ ID NO: 114 - DNA, 10 bp, Unknown
  This is missing from Primer #5
ggtactaagg

SEQ ID NO: 115 - DNA, 13 bp, Mus musculus
tttttttttttac

SEQ ID NO: 116 - DNA, 13 bp, Ratus ratus
tttttttttttca

SEQ ID NO: 117 - DNA, 13 bp, Mus musculus
tttttttttttcc

SEQ ID NO: 118 - DNA, 13 bp, Mus musculus
tttttttttttcg

SEQ ID NO: 119 - DNA, 13 bp, Mus musculus
tttttttttttgc

SEQ ID NO: 120 - DNA, 23 bp, E. coli
taatacgactcactatagggccc

SEQ ID NO: 121 - DNA, 18 bp, Mus musculus
ctcttatctcggcatttg

SEQ ID NO: 122 - DNA, 18 bp, Mus musculus
ggacaagagaaagcagcg

SEQ ID NO: 123 - DNA, 18 bp, Mus musculus
cgatgccgaagaacaaga

SEQ ID NO: 124 - DNA, 18 bp, Mus musculus
ctgcctgtcttgtcttcg

SEQ ID NO: 125 - DNA, 20 bp, Mus musculus
gaaaagcaagaggaaagatt

SEQ ID NO: 126 - DNA, 20 bp, Mus musculus
aagtcttcgaatgatgagaa

SEQ ID NO: 127 - DNA, 20 bp, Mus musculus
aatgacctccaccagcagct

SEQ ID NO: 128 - DNA, 20 bp, Mus musculus
tctcaggtacacagtgatcc

SEQ ID NO: 129 - DNA, 22 bp, Mus musculus
acatgagccccccagaagtacg

SEQ ID NO: 130 - DNA, 22 bp, Mus musculus
atgaccggaggaggagggagaa

SEQ ID NO: 131 - DNA, 20 bp, Mus musculus
cagaggaaagagagaagtcc

SEQ ID NO: 132 - DNA, 19 bp, Mus musculus
cacacggtgacagtgctgg

SEQ ID NO: 133 - DNA, 22 bp, Mus musculus
cctgagtatgcggtcctgactt

SEQ ID NO: 134 - DNA, 22 bp, Mus musculus
ccgggtctcccggtctggccga

SEQ ID NO: 135 - DNA, 22 bp, Mus musculus
gaaagcatgatccgcgacgtgg

SEQ ID NO: 136 - DNA, 23 bp, Mus musculus
gtagacctgcccggactccgcaa

SEQ ID NO: 137 - DNA, 21 bp, Mus musculus
atagcacagcttccctttgat

SEQ ID NO: 138 - DNA, 20 bp, Mus musculus
aacatgcattgttaccaact

SEQ ID NO: 139 - DNA, 27 bp, Homo sapien
attcgagttgtccacagtcagcaatgg

SEQ ID NO: 140 - DNA, 28 bp, Homo sapien
atggtcaacccccaccgtgttcttcgac

SEQ ID NO: 141 - DNA, 31 bp, Artificial Sequence
  Probe for gel mobility shift assays and competition experiments
gatcctagaagggaaaccgaaactgaggatc

SEQ ID NO: 142 - DNA, 30 bp, Artificial Sequence
  Probe for gel mobility shift assays and competition experiments
gatcgaggggaaaagtgaaagacacgggca

SEQ ID NO: 143 - DNA, 30 bp, Artificial Sequence
  Probe for gel mobility shift assays and competition experiments
gatcgaggggaaaagataaagacacgggca

SEQ ID NO: 144 - DNA, 30 bp, Artificial Sequence
  Probe for gel mobility shift assays and competition experiments
gatcgaggggaaaagtggctgacacgggca

SEQ ID NO: 145 - DNA, 29 bp, Artificial Sequence
  Probe for gel mobility shift assays and competition experiments
gatcattataaaaaaagtgcgaacgcgag